(19) United States
(12) Patent Application Publication
(10) Pub. No.: US 2023/0416821 A1
(43) Pub. Date: Dec. 28, 2023

(54) METHODS AND COMPOSITIONS FOR PROBE DETECTION AND READOUT SIGNAL GENERATION

(71) Applicant: 10x Genomics, Inc., Pleasanton, CA (US)

(72) Inventors: Felice Alessio BAVA, Rome (IT); Marco Mignardi, Sundbyberg (SE)

(21) Appl. No.: 18/300,814

(22) Filed: Apr. 14, 2023

Related U.S. Application Data
(60) Provisional application No. 63/331,742, filed on Apr. 15, 2022.

(51) Int. Cl. C12Q 1/6881; C12Q 1/6841

(52) U.S. Cl. CPC C12Q 1/6881; C12Q 1/6841

(57) ABSTRACT

In some aspects disclosed herein are methods and compositions for detecting an analyte such as a target nucleic acid in a biological sample, said method comprising generating and analyzing a detectable signal associated with the target nucleic acid and a separate signal associated with a region of interest in the target nucleic acid.

### CROSS-REFERENCE TO RELATED APPLICATION

This application claims priority to U.S. Provisional Patent Application No. 63/331,742, filed Apr. 15, 2022, entitled “METHODS AND COMPOSITIONS FOR PROBE DETECTION AND READOUT SIGNAL GENERATION,” which is herein incorporated by reference in its entirety for all purposes.

### FIELD

The present disclosure generally relates to methods and compositions for detection of a target analyte in a sample.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, in situ assays are important tools, for example, for understanding the molecular basis of cell identity and developing treatment for diseases. Certain existing in situ analyte detection methods can yield detection signals outside of an optimal range. For example, signals with small size and/or weak intensity may not reach a threshold of detection. Alternatively, signals with large size and/or high intensity may result in optical overcrowding, especially when in close proximity. In either case, sensitivity can be compromised, reducing the quality of image analysis. There is a need for new and improved methods for in situ assays. The present disclosure addresses these and other needs.

### SUMMARY

In some embodiments, provided herein is a method for analyzing a biological sample, comprising: a) contacting the biological sample with a plurality of encoding probes, wherein: the plurality of encoding probes comprises a first encoding probe and one or more second encoding probes, the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, and each second encoding probe is circular or circularizable; b) circularizing the first encoding probe to generate a circularized first encoding probe, and optionally circularizing the one or more second encoding probes; c) contacting the biological sample with one or more primary detectable probes that hybridize to the first encoding probe and/or the second encoding probe; d) detecting a signal associated with the one or more primary detectable probes; e) generating a rolling circle amplification (RCA) product of the circularized first encoding probe; and f) detecting a signal associated with the RCA product.

In any of the embodiments herein, the interrogatory region can be complementary to the region of interest, and hybridization of the interrogatory region to the region of interest can allow ligation to circularize the first encoding probe using the first target sequence as template. In any of the embodiments herein, the first encoding probe can be circularized with gap filling prior to the ligation. In any of the embodiments herein, the first encoding probe can be circularized without gap filling prior to the ligation. In any of the embodiments herein, upon hybridization to the target nucleic acid, a flap region on the 5′ and/or 3′ end of the first encoding probe can formed, and the flap region or a portion thereof can be cleaved prior to the ligation. In any of the embodiments herein, upon hybridization to the target nucleic acid, a flap region on the 5′ and/or 3′ end of the first encoding probe does not need to be formed.

In any of the embodiments herein, the first encoding probe can comprise one or more ribonucleotides. In any of the embodiments herein, the one or more second encoding probes can comprise one or more ribonucleotides. In any of the embodiments herein, the first encoding probe and/or one or more second encoding probes can comprise a ribonucleotide at a 3′ ligatable end. In any of the embodiments herein, the first encoding probe and/or one or more second encoding probes can be composed primarily of DNA. In any of the embodiments herein, the first encoding probe, circularized first encoding probe and/or one or more second encoding probes can each comprise less than five consecutive ribonucleotides. In any of the embodiments herein, the target nucleic acid can comprise RNA.

In any of the embodiments herein, the biological sample can comprises a counterpart target nucleic acid comprising the first target sequence except that the region corresponding to the region of interest is not complementary to the interrogatory region in the first encoding probe, thereby not allowing ligation of the ends of the first encoding probe using the counterpart target nucleic acid as template. In any of the embodiments herein, the biological sample can comprise a counterpart target nucleic acid comprising a counterpart first target sequence having the same sequence as the first target sequence except that the region of the counterpart first target sequence corresponding to the region of interest is not complementary to the interrogatory region in the first encoding probe, thereby not allowing ligation of the ends of the first encoding probe using the counterpart target nucleic acid as template.

In any of the embodiments herein, the first encoding probe can comprise a first hybridization region complementary to the first target sequence or a portion thereof. In any of the embodiments herein, the first hybridization region can be a split hybridization region comprising a 5′ hybridization region and a 3′ hybridization region. In any of the embodiments herein, the interrogatory region can be in the 5′ hybridization region or the 3′ hybridization region. In any of the embodiments herein, the interrogatory region can be at the 5′ end or the 3′ end of the first encoding probe, optionally wherein the interrogatory region comprises a 5′ or a 3′ terminal nucleotide.

In any of the embodiments herein, the region of interest can be a single nucleotide of interest or a dinucleotide of interest. In any of the embodiments herein, the region of interest can be selected from the group consisting of a single-nucleotide polymorphism (SNP), a single-nucleotide variant (SNV), a single-nucleotide substitution, a point mutation, a single-nucleotide insertion, and a single-nucleotide deletion. In any of the embodiments herein, the target nucleic acid can be an mRNA and the region of interest can comprise an exon-intron junction in a pre-mRNA or an exon-exon junction in a spliced mRNA.

In any of the embodiments herein, each second encoding probe can comprise a second hybridization region complementary to the second target sequence or a portion thereof. In any of the embodiments herein, the biological sample can be contacted with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more second encoding probes each hybridizing to a different second target sequence in the target nucleic acid. In any of the embodiments herein, the first target sequence can be 3′ or 5′ to the second target sequences in the target nucleic acid, or the first target sequence can be between two adjacent second target sequences in the target nucleic acid. In any of the embodiments herein, the first target sequence can be 3′ or 5′ to the second target sequences in the target nucleic acid, or the first target sequence can be between two second target sequences in the target nucleic acid. In any of the embodiments herein, the first encoding probe and the one or more second encoding probes can be independently a single molecule or provided in multiple molecules. In any of the embodiments herein, the first encoding probe and the one or more second encoding probes can be each independently provided as a single molecule or provided as multiple molecules.

In any of the embodiments herein, the first encoding probe and/or the one or more second encoding probes independently can comprise one or more barcode regions. In any of the embodiments herein, the first encoding probe and/or the one or more second encoding probes can each independently comprise one or more barcode regions. In any of the embodiments herein, the first encoding probe can comprise one or more barcode sequences that are not present in the one or more second encoding probes. In any of the embodiments herein, the one or more second encoding probes and optionally the first encoding probe can collectively comprise a combination of hybridization barcode sequences that correspond to the target nucleic acid. In any of the embodiments herein, the one or more second encoding probes and optionally the first encoding probe can comprise a combination of hybridization barcode sequences that corresponds to the target nucleic acid. In any of the embodiments herein, at least one of the one or more second encoding probes and optionally the first encoding probe does not need to comprise a particular hybridization barcode sequence in the combination of hybridization barcode sequences. In any of the embodiments herein, at least one of the one or more second encoding probes and optionally the first encoding probe does not need to comprise any hybridization barcode sequence in the combination of hybridization barcode sequences. In any of the embodiments herein, the first encoding probe can comprise an amplifiable barcode sequence, and the RCA product of the circularized first encoding probe can comprise multiple copies of the complement of the amplifiable barcode sequence, optionally wherein the amplifiable barcode sequence is determined by sequencing by ligation, sequencing by synthesis, sequencing by hybridization, or a combination thereof. In any of the embodiments herein, the primary detectable probes can be hybridized, in sequential cycles, to the hybridization barcode sequences in the first encoding probe and/or the one or more second encoding probes. In any of the embodiments herein, a temporal order of signals detected in the sequential cycles associated with the primary detectable probes can correspond to the combination of hybridization barcode sequences which corresponds to the target nucleic acid.

In any of the embodiments herein, the one or more second encoding probes and/or the first encoding probe can comprise one or more hybridization barcode sequences that correspond to the target nucleic acid. In any of the embodiments herein, at least one of the one or more second encoding probes and/or the first encoding probe does not need to comprise a particular hybridization barcode sequence in the one or more hybridization barcode sequences. In any of the embodiments herein, at least one of the one or more second encoding probes and/or the first encoding probe does not need to comprise any hybridization barcode sequence in the one or more hybridization barcode sequences. In any of the embodiments herein, the first encoding probe can comprise an amplifiable barcode sequence, and the RCA product of the circularized first encoding probe can comprise multiple copies of the complement of the amplifiable barcode sequence. In any of the embodiments herein, the primary detectable probes can be hybridized, in sequential cycles, to the hybridization barcode sequences in the one or more second encoding probes and/or the first encoding probe. In any of the embodiments herein, a temporal order of signals detected in the sequential cycles associated with the primary detectable probes can correspond to the one or more hybridization barcode sequences which correspond to the target nucleic acid.

In any of the embodiments herein, the first encoding probe can comprise an amplifiable barcode sequence corresponding to the target nucleic acid or a portion thereof, optionally wherein the amplifiable barcode sequence in the first encoding probe corresponds to the first target sequence or a portion thereof, optionally wherein the amplifiable barcode sequence is determined by sequencing by ligation, sequencing by synthesis, sequencing by hybridization, or a combination thereof. In any of the embodiments herein, the first encoding probe can comprise an amplifiable barcode sequence corresponding to the first target sequence or a portion thereof and/or the target nucleic acid or a portion thereof. In any of the embodiments herein, the first encoding probe can comprise a further amplifiable barcode sequence corresponding to the region of interest, optionally wherein the amplifiable barcode sequence and the further amplifiable barcode sequence can be the same or different sequences and can be overlapping or non-overlapping, optionally wherein the further amplifiable barcode sequence is determined by sequencing by ligation, sequencing by synthesis, sequencing by hybridization, or a combination thereof. In any of the embodiments herein, the first encoding probe can comprise a further amplifiable barcode sequence corresponding to the region of interest, wherein the amplifiable barcode sequence and the further amplifiable barcode sequence are the same or different sequences and are overlapping or non-overlapping. In any of the embodiments herein, each second encoding probe can comprise an amplifiable barcode sequence corresponding to the target nucleic acid or a portion thereof, optionally wherein the amplifiable barcode sequence in each second encoding probe corresponds to the second target sequence or a portion thereof, optionally wherein the amplifiable barcode sequence in each second encoding probe is determined by sequencing by ligation, sequencing by synthesis, sequencing by hybridization, or a combination thereof. In any of the embodiments herein, each second encoding probe can comprise an amplifiable barcode sequence corresponding to the second target sequence or a portion thereof and/or the target nucleic acid or a portion thereof. In any of the embodiments herein, the amplifiable barcode sequence of the first encoding probe, the further amplifiable barcode sequence of the first encoding probe, and/or the amplifiable barcode sequence of each second encoding probe, can be determined by sequencing by ligation, sequencing by synthesis, sequencing by hybridization, or a combination thereof.

In any of the embodiments herein, independently each primary detectable probe can be fluorescently labeled or can hybridize to a fluorescently labeled probe. In any of the embodiments herein, independently each primary detectable probe can directly or indirectly hybridize to a hybridization barcode sequence in the first encoding probe and/or the one or more second encoding probes. In any of the embodiments herein, independently each primary detectable probe can be fluorescently labeled or hybridize to a fluorescently labeled probe, and independently each primary detectable probe can directly or indirectly bind to a hybridization barcode sequence in the first encoding probe and/or the one or more second encoding probes. In any of the embodiments herein, the primary detectable probes can be hybridized directly to the hybridization barcode sequences in the one or more second encoding probe.

In any of the embodiments herein, in step b), the first encoding probe can be circularized and the one or more second encoding probes does not need to be circularized. In any of the embodiments herein, in step b), the first encoding probe and the one or more second encoding probes can be circularized. In any of the embodiments herein, the method can comprise performing steps b), c), and d) in that order. In any of the embodiments herein, the method can comprise performing step e) before steps c), d) and f).

In any of the embodiments herein, the method can further comprise removing the one or more primary detectable probes without removing the first encoding probe and/or the one or more second encoding probes from the target nucleic acid. In any of the embodiments herein, the method can further comprise contacting the biological sample with another one or more primary detectable probes that hybridize to the first encoding probe and/or the one or more second encoding probes. In any of the embodiments herein, the one or more second encoding probes can be circularized using the corresponding second target sequence as template.

In any of the embodiments herein, the RCA product of the first encoding probe can be a first RCA product, and the circularized one or more second encoding probes can be used as template to generate one or more second RCA products. In any of the embodiments herein, the RCA product of the first encoding probe can be a first RCA product, and the circular or circularized one or more second encoding probes can be used as template to generate one or more second RCA products. In any of the embodiments herein, the method can further comprise detecting a signal associated with the one or more second RCA products. In any of the embodiments herein, the signal associated with the first RCA product and the signal associated with the one or more second RCA products can be detected simultaneously or sequentially in either order. In any of the embodiments herein, the first RCA product and/or the one or more second RCA products can be contacted with one or more secondary detectable probes that each directly or indirectly hybridizes to an RCA product. In any of the embodiments herein, the first RCA product and/or the one or more second RCA products can be contacted with one or more secondary detectable probes that each directly or indirectly binds to an RCA product. In any of the embodiments herein, independently each secondary detectable probe can directly or indirectly hybridize to a complementary barcode sequence in the first RCA product or the one or more second RCA products. In any of the embodiments herein, independently each secondary detectable probe can directly or indirectly bind to a complementary barcode sequence in the first RCA product or the one or more second RCA products. In any of the embodiments herein, the complementary barcode sequence in the first RCA product or the one or more second RCA products can be the complement of an amplifiable barcode sequence in the first encoding probe or the one or more second encoding probes.

In any of the embodiments herein, independently each secondary detectable probe can be fluorescently labeled or can hybridize to a fluorescently labeled probe. In any of the embodiments herein, the secondary detectable probes can be hybridized, in sequential cycles, to the complementary barcode sequences in the first RCA product or the one or more second RCA products. In any of the embodiments herein, a temporal order of signals detected in the sequential cycles associated with the secondary detectable probes can correspond to the complementary barcode sequence which corresponds to the target nucleic acid or a portion thereof.

In any of the embodiments herein, the method can comprise using signals associated with the first RCA product to select a first number of second encoding probes for RCA. In any of the embodiments herein, the method can further comprise using signals associated with the RCA products of the first number of second encoding probes to select a second number of second encoding probes for RCA. In any of the embodiments herein, two or more of the second encoding probes can comprise different primer binding sequences for RCA. In any of the embodiments herein, the method can comprise using signals associated with the one or more primary detectable probes to select a duration of RCA to generate the RCA product of the first encoding probe. In any of the embodiments herein, the method can comprise using signals associated with the first RCA product to select a number of second encoding probes for RCA and/or to select a duration of RCA to generate RCA product(s) of the one or more second encoding probes. In any of the embodiments therein, the method can comprise using signals associated with the RCA product of the first encoding probe to select a number of second encoding probes for RCA and/or to select a duration of RCA to generate RCA product(s) of the one or more second encoding probes.

In any of the embodiments herein, the RCA can be performed in situ in the biological sample. In any of the embodiments herein, products of the RCA can be formed in situ at one or more locations in the biological sample.

In some embodiments, provided herein is a method for analyzing a biological sample, comprising: a) contacting the biological sample with a plurality of encoding probes, wherein: the plurality of encoding probes comprises a first encoding probe, an alternative first encoding probe, and one or more second encoding probes, the first/alternative first encoding probe and each second encoding probe are capable of hybridizing to a first target sequence and a second target sequence, respectively, in a target nucleic acid in the biological sample, the first encoding probe and the alternative first encoding probe are circularizable and each comprises an interrogatory region for interrogating a region of interest in the first target sequence, wherein the interrogatory region of the first encoding probe is complementary to the region of interest and the interrogatory region of the alternative first encoding probe is not complementary to the region of interest, and each second encoding probe is circular or circularizable; b) circularizing the first encoding probe hybridized to the region of interest to generate a circularized first encoding probe using the first target sequence as template, wherein the alternative first encoding probe is not sufficiently complementary to the region of interest to allow circularization of the alternative first encoding probe; c) contacting the biological sample with one or more primary detectable probes that hybridize to the first/alternative first encoding probe and/or the second encoding probe; d) detecting a signal associated with the one or more primary detectable probes; e) generating a rolling circle amplification (RCA) product of the circularized first encoding probe; and f) detecting a signal associated with the RCA product.

In some embodiments, provided herein is a method for analyzing a biological sample, comprising: a) contacting the biological sample with a plurality of encoding probes, wherein: the plurality of encoding probes comprises a first encoding probe and one or more second encoding probes, the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, the region of interest is complementary to the interrogatory region, each second encoding probe is circular or circularizable, and the biological sample comprises a counterpart target nucleic acid comprising (i) a counterpart first target sequence having the same sequence as the first target sequence except that the region of the counterpart first target sequence corresponding to the region of interest is not complementary to the interrogatory region in the first encoding probe, and (ii) the second target sequence; b) circularizing the first encoding probe to generate a circularized first encoding probe using the first target sequence as template, and not using the counterpart first target sequence as template; c) contacting the biological sample with one or more primary detectable probes that hybridize to the first encoding probe and/or the one or more second encoding probe; d) detecting a signal associated with the one or more primary detectable probes; e) generating a rolling circle amplification (RCA) product of the circularized first encoding probe; and f) detecting a signal associated with the RCA product.

In some embodiments, provided herein is a method for analyzing a biological sample, comprising: a) contacting the biological sample with a plurality of encoding probes, wherein: the plurality of encoding probes comprises a first encoding probe, an alternative first encoding probe, and one or more second encoding probes, the first encoding probe and each second encoding probe are capable of hybridizing to a first target sequence and a second target sequence, respectively, in a target nucleic acid in the biological sample, the alternative first encoding probe and each second encoding probe are capable of hybridizing to a counterpart first target sequence and the second target sequence, respectively, in a counterpart target nucleic acid in the biological sample, the first encoding probe is circularizable and comprises an interrogatory region for interrogating a region of interest in the first target sequence, the alternative first encoding probe is circularizable and comprises an alternative interrogatory region for interrogating a counterpart region of interest in the counterpart first target sequence, and each second encoding probe is circular or circularizable; b) circularizing the first encoding probe to generate a circularized first encoding probe using the first target sequence as template, and circularizing the alternative first encoding probe to generate a circularized alternative first encoding probe using the counterpart first target sequence as template; c) contacting the biological sample with one or more primary detectable probes that hybridize to the first encoding probe, the alternative first encoding probe, and/or the second encoding probe; d) detecting a signal associated with the one or more primary detectable probes; e) generating a rolling circle amplification (RCA) product of the circularized first encoding probe and an RCA product of the circularized alternative first encoding probe; and f) detecting a signal associated with the RCA product of the circularized first encoding probe and/or a signal associated with the RCA product of the circularized alternative first encoding probe.

In any of the embodiments herein, the counterpart first target sequence can comprise the sequence of the first target sequence except that the counterpart region of interest is different than the region of interest. In any of the embodiments herein, the counterpart first target sequence can have the same sequence as the first target sequence except that the counterpart region of interest is different than the region of interest. In any of the embodiments herein, the region of interest and the counterpart region of interest can be different alleles of the group consisting of a single-nucleotide polymorphism (SNP), a single-nucleotide variant (SNV), a single-nucleotide substitution, a point mutation, a single-nucleotide insertion, and a single-nucleotide deletion. In any of the embodiments herein, the first encoding probe does not need to be circularized using the counterpart first target sequence as template, and/or the alternative first encoding probe does not need to be circularized using the first target sequence as template. In any of the embodiments herein, the first encoding probe and the alternative first encoding probe can comprise different barcode regions. In any of the embodiments herein, signals associated with the RCA product of the circularized first encoding probe and signals associated with the RCA product of the circularized alternative first encoding probe can be detected in different locations of the biological sample.

In some embodiments, provided herein is a method for analyzing a biological sample, comprising: a) contacting the biological sample with a plurality of encoding probes or probe sets each capable of hybridizing to a target sequence in a target nucleic acid in the biological sample; b) ligating the ends of the encoding probes or probes sets to form a ligation product; c) contacting the biological sample with a plurality of detectable probes that hybridize to the plurality of encoding probes or probe sets, wherein each encoding probe or probe set forms an amplification complex with two or more detectable probes; and d) detecting a signal associated with the two or more detectable probes.

In any of the embodiments herein, an encoding probe set of the plurality of encoding probes or probe sets can comprise a first probe and a second probe. In any of the embodiments herein, the first and/or second probe can comprise an overhang that is for directly or indirectly binding to the detectable probes, and that is non-hybridizing to the target nucleic acid. In some of any of the embodiments herein, the first and/or second probe can comprises an overhang that is for directly or indirectly binding to the detectable probes, and that does not hybridize to the target nucleic acid. In any of the embodiments herein, b) can comprise two ligations to join the first and second probes at both ends. In any of the embodiments herein, each of the detectable probes can comprise a region for directly or indirectly binding to two or more fluorescently labeled probes. In any of the embodiments herein, the plurality of encoding probes or probe sets can comprise one or more circularizable encoding probes. In any of the embodiments herein, the one or more circularizable encoding probes can be ligated with gap filling prior to the ligation or without gap filling prior to the ligation. In any of the embodiments herein, the one or more circularizable encoding probes can each comprises one or more ribonucleotides, and optionally the one or more circularizable encoding probes can each comprise a ribonucleotide at a 3′ ligatable end. In any of the embodiments herein, upon hybridization to the target nucleic acid, a flap region on the 5′ and/or 3′ end of the one or more circularizable encoding probes can be formed, and the flap region or a portion thereof can be cleaved prior to the ligation. In any of the embodiments herein, upon hybridization to the target nucleic acid, a flap region on the 5′ and/or 3′ end of the one or more circularizable encoding probes does not need to be formed.

In any of the embodiments herein, the method can be performed in situ in the biological sample. In any of the embodiments herein, one or more of the signals can be detected in situ in the biological sample.

In some embodiments, provided herein is a kit for analyzing a biological sample, comprising: a) a plurality of encoding probes comprising a first encoding probe and one or more second encoding probes, wherein: the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, and each second encoding probe is circular or circularizable; and b) one or more primary detectable probes that hybridize to the first encoding probe and/or the second encoding probe. In any of the embodiments herein, the kit can further comprise a ligase for circularizing the first encoding probe to generate a circularized first encoding probe, and optionally for circularizing one or more second encoding probes to generate one or more circularized second encoding probes. In any of the embodiments herein, the kit can further comprise one or more secondary detectable probes that directly or indirectly hybridize to an RCA product of the first encoding probe or an RCA product of one or more second encoding probes. In any of the embodiments herein, the kit can further comprise one or more secondary detectable probes that directly or indirectly bind to an RCA product of the first encoding probe or an RCA product of one or more second encoding probes. In any of the embodiments herein, the one or more second encoding probes of the kit can comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more second encoding probes that each hybridize to a different second target sequence in the target nucleic acid.

In some embodiments, provided herein is a method for analyzing a biological sample, comprising: a) contacting the biological sample with a plurality of encoding probes, wherein: the plurality of encoding probes comprises a first encoding probe and one or more second encoding probes, the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, and each second encoding probe is circular or circularizable; b) contacting the biological sample with one or more primary detectable probes that hybridize to the first encoding probe and/or the second encoding probe; c) detecting a signal associated with the one or more primary detectable probes; d) circularizing the first encoding probe to generate a circularized first encoding probe; e) generating a rolling circle amplification (RCA) product of the circularized first encoding probe; and f) detecting a signal associated with the RCA product.

In some embodiments, provided herein is a method for analyzing a biological sample, comprising: a) contacting the biological sample with a plurality of encoding probes, wherein: the plurality of encoding probes comprises a first encoding probe, an alternative first encoding probe, and one or more second encoding probes, the first/alternative first encoding probe and each second encoding probe are capable of hybridizing to a first target sequence and a second target sequence, respectively, in a target nucleic acid in the biological sample, the first encoding probe and the alternative first encoding probe are circularizable and each comprises an interrogatory region for interrogating a region of interest in the first target sequence, wherein the interrogatory region of the first encoding probe is complementary to the region of interest and the interrogatory region of the alternative first encoding probe is not, and each second encoding probe is circular or circularizable; b) contacting the biological sample with one or more primary detectable probes that hybridize to the first/alternative first encoding probe and/or the second encoding probe; c) detecting a signal associated with the one or more primary detectable probes; d) circularizing the first encoding probe hybridized to the region of interest to generate a circularized first encoding probe using the first target sequence as template, wherein the alternative first encoding probe is not hybridized to the region of interest to allow circularization of the alternative first encoding probe; e) generating a rolling circle amplification (RCA) product of the circularized first encoding probe; and f) detecting a signal associated with the RCA product.

In some embodiments, provided herein is a method for analyzing a biological sample, comprising: a) contacting the biological sample with a plurality of encoding probes, wherein: the plurality of encoding probes comprises a first encoding probe and one or more second encoding probes, the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, the region of interest is complementary to the interrogatory region, each second encoding probe is circular or circularizable, and the biological sample comprises a counterpart target nucleic acid comprising (i) a counterpart first target sequence which comprises the sequence of the first target sequence except that the region corresponding to the region of interest is not complementary to the interrogatory region in the first encoding probe, and (ii) the second target sequence; b) contacting the biological sample with one or more primary detectable probes that hybridize to the first encoding probe and/or the second encoding probe; c) detecting a signal associated with the one or more primary detectable probes; d) circularizing the first encoding probe to generate a circularized first encoding probe using the first target sequence as template, and not using the counterpart first target sequence as template; e) generating a rolling circle amplification (RCA) product of the circularized first encoding probe; and f) detecting a signal associated with the RCA product.

In some embodiments, provided herein is a method for analyzing a biological sample, comprising: a) contacting the biological sample with a plurality of encoding probes, wherein: the plurality of encoding probes comprises a first encoding probe, an alternative first encoding probe, and one or more second encoding probes, the first encoding probe and each second encoding probe are capable of hybridizing to a first target sequence and a second target sequence, respectively, in a target nucleic acid in the biological sample, the alternative first encoding probe and each second encoding probe are capable of hybridizing to a counterpart first target sequence and the second target sequence, respectively, in a counterpart target nucleic acid in the biological sample, the first encoding probe is circularizable and comprises an interrogatory region for interrogating a region of interest in the first target sequence, the alternative first encoding probe is circularizable and comprises an alternative interrogatory region for interrogating a counterpart region of interest in the counterpart first target sequence, and each second encoding probe is circular or circularizable; b) contacting the biological sample with one or more primary detectable probes that hybridize to the first encoding probe, the alternative first encoding probe, and/or the second encoding probe; c) detecting a signal associated with the one or more primary detectable probes; d) circularizing the first encoding probe to generate a circularized first encoding probe using the first target sequence as template, and circularizing the alternative first encoding probe to generate a circularized alternative first encoding probe using the counterpart first target sequence as template; e) generating a rolling circle amplification (RCA) product of the circularized first encoding probe and an RCA product of the circularized alternative first encoding probe; and f) detecting a signal associated with the RCA product of the circularized first encoding probe and/or a signal associated with the RCA product of the circularized alternative first encoding probe.

### BRIEF DESCRIPTION OF THE DRAWINGS

The drawings illustrate certain features and advantages of this disclosure. These embodiments are not intended to limit the scope of the appended claims in any manner.

FIG. 1 shows a schematic illustrating exemplary detection of a first encoding probe and a second encoding probe hybridized to a first and second target sequence, respectively, of a target nucleic acid.

FIG. 2A shows schematics illustrating exemplary sequential detection of smFISH and RCA signals from a target nucleic acid using a first encoding probe and a plurality of second encoding probes.

FIG. 2B shows schematics illustrating various exemplary encoding probe or encoding probe set configurations for signal amplification and detection.

FIG. 3 shows schematics illustrating exemplary sequential detection of RCA signals from a target nucleic acid using a first encoding probe, a first number of second encoding probes, and a second number of second encoding probes.

FIG. 4 shows a schematic illustrating a first encoding probe hybridized to a first target sequence comprising a region of interest (e.g., SNP1) in a target nucleic acid, and an alternative first encoding probe hybridized to a counterpart first target sequence comprising a counterpart first region of interest (e.g., SNP2) in a counterpart target nucleic acid.

FIG. 5 shows a schematic illustrating an exemplary workflow for detecting a signal associated with a target nucleic acid in a biological sample using a plurality of circularizable probes (e.g., padlock probes).

FIG. 6 shows a schematic illustrating an exemplary workflow for detecting a signal associated with a target nucleic acid, and a signal associated with a region of interest in the target nucleic acid, in a biological sample.

FIG. 7 shows a schematic illustrating an exemplary workflow for generating and detecting tunable signals associated with a target nucleic acid and a region of interest in the target nucleic acid in a biological sample.

FIG. 8 shows results of signals associated with a target nucleic acid Polr2a detected using padlock probes hybridized with single-labeled and double-labeled detection probes as described in Example 1.

FIG. 9 shows results of signals associated with a target nucleic acid Polr2a detected using padlock probes hybridized with single-labeled and double-labeled detection probes as described in Example 1.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some aspects, the present disclosure provides methods and compositions for both sensitive detection of target nucleic acids and detection of variants of target nucleic acids with single-nucleotide resolution (e.g. to identify mutations, single-nucleotide polymorphisms (SNPs) or splice variants). In some aspects, the present disclosure provides methods and compositions for tuning the intensity of a signal associated with a target nucleic acid. Tuning of signal intensity may depend on the level of autofluorescence in a biological sample in which a nucleic acid is detected. In some embodiments, provided herein are probes or probe sets (e.g., linear and/or circularizable) that hybridize to the target nucleic acid and that can be bound by detectable probes, e.g. detection oligonucleotides (DOs), such that an amplified signal associated with the target nucleic acid can be detected.

In an exemplary method of the methods provided herein, a target nucleic acid (e.g. a specific transcript) in a biological sample is hybridized by a number (e.g. 20-30) of circularizable probes or probe sets (e.g. padlock probes). Some of the probes can be designed to target specific sites on the transcript (e.g. point mutations and splice variants). One or more probes may comprise specific sequences such as barcode regions. Barcode regions can allow for probe-specific detection and processing. For example, barcode regions can be hybridized by specific detectable probes (such as the primary detectable probes or secondary detectable probes, as described herein). In some cases, different probes or different sets of probes may each comprise a specific sequence (e.g., unique to the probe or set of probes) for binding specific primers to initiate RCA. In some embodiments, circularizable probes or probe sets are circularized on and/or around the target nucleic acid. The circularized probes can be labeled with the primary detectable probes, e.g. detection oligonucleotides (DOs). Detection oligonucleotides such as primary detectable probes may be labeled with one or more fluorophores. The sample is imaged to detect a signal associated with the labeled circularized probes. After imaging, the detection oligonucleotides such as primary detectable probes are stripped. The stripping may be performed under stringent conditions. In some cases, probe-specific primers are hybridized to circularized probes of interest and rolling circle amplification (RCA) is performed to produce one or more RCA products, and the RCA products are detected (e.g. using detectable probes, such as the secondary detectable probes, and imaging). After imaging of RCA products, one or more additional rounds of RCA can be performed. Additional rounds of RCA may amplify circularized probes that were not amplified in the first RCA round, and/or RCA may be allowed to proceed for a different period of time in order to obtain a desired signal intensity (e.g. a longer period of time for a stronger signal, or a shorter period of time for a weaker signal). Whether or not to perform a first or additional round of RCA may depend on the quality of the signal obtained in a previous step. For example, if a detected signal is too weak, e.g., sample background or autofluorescence is too high after detecting the circularized probes with primary detectable probes, a first round of RCA can be performed and the resulting RCA products can be detected, e.g. using secondary detectable probes. The sample can be imaged and the user can decide whether to perform an additional round of RCA. In some embodiments, the probe designs provided herein allow flexibility for signal generation and degree of signal amplification.

In some embodiments, a target nucleic acid in a sample, such as a target RNA (e.g. target transcript) is hybridized by one or more encoding probes (e.g. circularizable encoding probes), which may be tiled across the transcript. In some instances, the circularizable encoding probes are ligated and circularized while hybridized to the transcript. The encoding probes can further comprise a backbone sequence (e.g. the sequence of the probe not directly hybridized to the transcript). The backbone sequences of two or more encoding probes can be the same or different. The backbone sequences may comprise barcode regions. The barcode regions of some probes may correspond to, or be used to identify specific target sequences or variations in the transcript, for example point mutations, single-nucleotide polymorphisms, or splice variants.

Any of the encoding probes may be “decorated” (e.g., labeled, hybridized, or bound) by detectable probes (e.g. the primary detectable probes provided herein). The primary detectable probes may be fluorescently labeled. Each primary detectable probe may comprise one or more fluorophores, and each encoding probe or probe set (e.g., circularized encoding probe) may be labeled by one or more primary detectable probes. In some embodiments, detectable probes can be fluorescently labeled and/or hybridized to a fluorescently labeled probe. In some embodiments, not all probes are decorated (e.g. some are kept “dark”). In some embodiments, the sample is imaged, and the position of the detected transcripts is recorded. In some embodiments, any of the encoding probes may be hybridized directly by fluorescently labeled detectable probes. The primary detectable probes can be stripped, and stripping may be performed under stringent conditions. In some embodiments, the encoding probes are labeled, imaged, and optionally stripped for another round of detection. Multiple rounds of detection using different sets of primary detectable probes may facilitate decoding of the identities of target nucleic acids, for example in multiplexed assays. A final round of stripping can be performed, for example before subsequent processing steps.

In some embodiments, probe-specific RCA primers are hybridized to one or more circularized encoding probes, and may be hybridized to specific encoding probes (e.g. an encoding probe targeting a SNP) or sets of encoding probes (e.g. a number of secondary encoding probes). RCA products can be generated and detected. The detection of a specific RCA product may be indicative of a transcript in general, and/or a variant of or within the transcript, e.g. a point mutation, splice variant, or SNP. After imaging of RCA products, one or more additional rounds of RCA may be performed. Whether or not to perform a first or additional rounds of RCA may depend on the quality of the signal obtained in a previous step. For example, if sample background or autofluorescence is too high after detecting the circularized encoding probes, a first round of RCA can be performed. The sample can be imaged and the user can decide whether to perform an additional round of RCA.

In some aspects, provided herein is a method for analyzing a biological sample. In some aspects, the method comprises contacting the biological sample with a plurality of encoding probes, wherein the plurality of encoding probes comprises a first encoding probe and one or more second encoding probes. In some embodiments, the first encoding probe and each second encoding probe are capable of hybridizing to a first target sequence and a second target sequence, respectively, in a target nucleic acid in the biological sample. In some embodiments, the first encoding probe is circularizable and comprises an interrogatory region for interrogating a region of interest in the first target sequence, and each second encoding probe is circular or circularizable. In some aspects, the method comprises contacting the biological sample with one or more primary detectable probes that hybridize to the first encoding probe and/or the second encoding probe and detecting a signal associated with the one or more primary detectable probes. In some aspects, the method comprises circularizing the first encoding probe to generate a circularized first encoding probe. In some embodiments, the first encoding probe is circularized using the first target sequence as template. In some aspects, the method comprises generating a rolling circle amplification (RCA) product of the circularized first encoding probe and detecting a signal associated with the RCA product. In some embodiments, an encoding probe, such as the first encoding probe, may be provided as an encoding probe set. An encoding probe set may comprise two or more probes, such as a first and second probe. An encoding probe set may be circularized upon ligation, or may remain linear. In some aspects, the method comprises generating a hybridization complex, such as an amplification complex, comprising the ligated encoding probe and a plurality of detectable probes. In some embodiments, the plurality of detectable probes can comprise one or more detectable probes that hybridize to the encoding probe, and one or more detectable probes that are configured to hybridize to the one or more detectable probes that are hybridized to the encoding probe. In some embodiments, the method comprises detecting a signal associated with the hybridization complex.

In some embodiments, the primary detectable probes described herein are probes that bind (e.g. hybridize) to encoding probes, which in turn hybridize to the target nucleic acid. Thus, in some embodiments, the primary detectable probe is not necessarily a primary nucleic acid probe that hybridizes directly to the target nucleic acid to be detected, and the primary detectable probe can be a secondary nucleic acid probe or higher order nucleic acid probe. The term “primary” in primary detectable probe as used herein is not intended to construe the order of the nucleic acid probe (e.g. as in a primary, secondary, or tertiary nucleic acid probe). In some embodiments, the primary detectable probes described herein are probes that bind (e.g. hybridize) directly to encoding probes. In some embodiments, the primary detectable probes described herein are probes that bind (e.g. hybridize) indirectly to encoding probes.

In some embodiments, the secondary detectable probes described herein are probes that indirectly or directly bind (e.g. hybridize) to an amplification product of an encoding probe, such as an RCA product. Thus, in some aspects, the secondary detectable probe can be a secondary nucleic acid probe or higher order nucleic acid probe. The term “secondary” in secondary detectable probe as used herein is not intended to construe the order of the nucleic acid probe (e.g. as in a primary, secondary, or tertiary nucleic acid probe). In some embodiments, the secondary detectable probes described herein are probes that bind (e.g. hybridize) directly to an amplification product of the encoding probe. In some embodiments, the secondary detectable probes described herein are probes that bind (e.g. hybridize) indirectly to an amplification product of the encoding probe.

In some embodiments, the interrogatory region is complementary to the region of interest. In some aspects, the hybridization of the interrogatory region to the region of interest allows ligation to circularize the first encoding probe using the first target sequence as template. In some embodiments, the interrogatory region is complementary to the region of interest, and following hybridization of the interrogatory region to the region of interest, the first encoding probe is configured to be ligated to circularize the first encoding probe using the first target sequence as template. In some embodiments, the first encoding probe is circularized with gap filling prior to the ligation. In some embodiments, the first encoding probe is circularized without gap filling prior to the ligation.

In some embodiments, upon hybridization to the target nucleic acid, a flap region on the 5′ and/or 3′ end of the first encoding probe is formed, and the flap region or a portion thereof is cleaved prior to the ligation. In some embodiments, upon hybridization to the target nucleic acid, a flap region on the 5′ and/or 3′ end of the first encoding probe is not formed.

In some embodiments, an encoding probe comprises a ribonucleotide, which may be at a 3′ ligatable end of the encoding probe. The encoding probe may be composed primarily of DNA (i.e. composed primarily of deoxyribonucleotides). For example, the encoding probe may be composed of more than 50%, more than 60%, more than 70% more than 80%, more than 90%, more than 95%, or more than 99% deoxyribonucleotides. In some embodiments, the encoding probe comprises no more than four consecutive ribonucleotides, either before or after ligation and/or circularization. In some embodiments, the target nucleic acid comprises RNA, and the inclusion of one or more ribonucleotides in an encoding probe can improve the efficiency and/or fidelity of ligation using the RNA as template.

In some embodiments, the biological sample comprises a counterpart target nucleic acid comprising a counterpart first target sequence having the same sequence as the first target sequence except that the region of the counterpart first target sequence corresponding to the region of interest is not complementary to the interrogatory region in the first encoding probe, thereby not allowing ligation of the ends of the first encoding probe using the counterpart target nucleic acid as template. In some embodiments, the first encoding probe is not circularized using as template a sequence that (i) has identity to the first target sequence, and (ii) does not comprise the region of interest and/or comprises a sequence that is different from the region of interest. In some embodiments, the biological sample comprises a counterpart target nucleic acid comprising a counterpart first target sequence having the same sequence as the first target sequence except that the region corresponding to the region of interest is not complementary to the interrogatory region in the first encoding probe. Thus, in some embodiments, the first encoding probe is not configured to be ligated and circularized using the counterpart target nucleic acid as template. In some embodiments, the first encoding probe is configured to be ligated and circularized using the target nucleic acid as template, and the first encoding probe is not configured to be ligated and circularized using the counterpart target nucleic acid as template.

In some embodiments, the first encoding probe comprises a first hybridization region complementary to the first target sequence or a portion thereof. The first hybridization region may be a split hybridization region comprising a 5′ hybridization region and a 3′ hybridization region. In some embodiments, the interrogatory region is in the 5′ hybridization region. In some embodiments, the interrogatory region is in the 3′ hybridization region. In some embodiments, the interrogatory region is at the 5′ end or the 3′ end of the first encoding probe. In some embodiments, the interrogatory region may comprise a 5′ or a 3′ terminal nucleotide of the encoding probe. In some embodiments, the interrogatory region does not comprise a 5′ or 3′ terminal nucleotide of the encoding probe.

The region of interest may be a single nucleotide of interest or a dinucleotide of interest, and may be selected from the group consisting of a single-nucleotide polymorphism (SNP), a single-nucleotide variant (SNV), a single-nucleotide substitution, a point mutation, a single-nucleotide insertion, and a single-nucleotide deletion. In some embodiments, the target nucleic acid is an mRNA and the region of interest comprises an exon-intron junction in a pre-mRNA or an exon-exon junction in a spliced mRNA.

In some aspects, each second encoding probe comprises a second hybridization region complementary to the second target sequence or a portion thereof. In some embodiments, the biological sample can be contacted with a plurality of second encoding probes, each hybridizing to a different second target sequence in the target nucleic acid. For example, the biological sample may be contacted with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more second encoding probes each hybridizing to a different second target sequence in the target nucleic acid. The first target sequence may be 3′ or 5′ to one or more second target sequences in the target nucleic acid. The first target sequence may be 3′ or 5′ to the second target sequences in the target nucleic acid, or the first target sequence may be between two second target sequences in the target nucleic acid.

In some embodiments, the first encoding probe and the one or more second encoding probes are each independently provided as a single molecule or provided as multiple molecules.

In some embodiments, the first encoding probe and the one or more second encoding probes each independently comprise one or more barcode regions. The first encoding probe may comprise one or more barcode sequences that are not present in the one or more second encoding probes. In some embodiments, the first encoding probe and/or one or more second encoding probes collectively comprise a combination of hybridization barcode sequences that correspond to the target nucleic acid. In some embodiments, at least one of the first encoding probe and/or one or more second encoding probes does not comprise a particular hybridization barcode sequence in the combination of hybridization barcode sequences. In some embodiments, at least one of the first encoding probe and/or one or more second encoding probes does not comprise any hybridization barcode sequence in the combination of hybridization barcode sequences. In some embodiments, the first encoding probe comprises an amplifiable barcode sequence. In some embodiments, the RCA product of the circularized first encoding probe comprises multiple copies of the complement of the amplifiable barcode sequence. In some embodiments, the amplifiable barcode sequence is determined by sequencing by ligation, sequencing by synthesis, sequencing by hybridization, or a combination thereof.

In some embodiments, the one or more second encoding probes and/or the first encoding probe comprise one or more hybridization barcode sequences that correspond to the target nucleic acid. In some embodiments, at least one of the one or more second encoding probes and/or the first encoding probe does not comprise a particular hybridization barcode sequence in the one or more hybridization barcode sequences. In some embodiments, at least one of the one or more second encoding probes and/or the first encoding probe does not comprise any hybridization barcode sequence in the one or more hybridization barcode sequences. In some embodiments, the first encoding probe comprises an amplifiable barcode sequence, and the RCA product of the circularized first encoding probe comprises multiple copies of the complement of the amplifiable barcode sequence. In some embodiments, the primary detectable probes are hybridized, in sequential cycles, to the hybridization barcode sequences in the one or more second encoding probes and/or the first encoding probe. In some embodiments, a temporal order of signals detected in the sequential cycles associated with the primary detectable probes corresponds to the one or more hybridization barcode sequences which correspond to the target nucleic acid.

In some embodiments, the primary detectable probes are hybridized, in sequential cycles, to the hybridization barcode sequences in the first encoding probe and the one or more second encoding probes. In some embodiments, a temporal order of signals detected in the sequential cycles associated with the primary detectable probes corresponds to the combination of hybridization barcode sequences which corresponds to the target nucleic acid.

In some embodiments, the first encoding probe comprises an amplifiable barcode sequence corresponding to the first target sequence or a portion thereof and/or the target nucleic acid or a portion thereof. The amplifiable barcode sequence may be determined by sequencing by ligation, sequencing by synthesis, sequencing by hybridization, or a combination thereof. In some embodiments, the first encoding probe comprises a further amplifiable barcode sequence corresponding to the region of interest. In some embodiments the amplifiable barcode sequence and the further amplifiable barcode sequence can be the same or different sequences. In some embodiments, the amplifiable barcode sequence and the further amplifiable barcode sequence can be overlapping or non-overlapping. In some embodiments, the further amplifiable barcode sequence is determined by sequencing by ligation, sequencing by synthesis, sequencing by hybridization, or a combination thereof.

The second encoding probe may comprise an amplifiable barcode sequence corresponding to the target nucleic acid or a portion thereof. In some embodiments the amplifiable barcode sequence in each second encoding probe corresponds to the second target sequence or a portion thereof. The amplifiable barcode sequence in each second encoding probe may be determined by sequencing by ligation, sequencing by synthesis, sequencing by hybridization, or a combination thereof.

In some embodiments, independently, each primary detectable probe is fluorescently labeled or hybridizes to a fluorescently labeled probe. Independently, each primary detectable probe may directly or indirectly bind to a hybridization barcode sequence in the first encoding probe and/or the one or more second encoding probes.

In some embodiments, the first encoding probe and the one or more second encoding probes are independently circular, circularized, or not circularized prior to or after detection of the signal associated with the one or more primary detectable probes. In some embodiments, the method comprises removing the one or more primary detectable probes (e.g. after detecting a signal associated with the one or more primary detectable probes). In some embodiments, the one or more primary detectable probes are removed without removing the first encoding probe and the one or more second encoding probes from the target nucleic acid. The method may further comprise contacting the biological sample with another one or more primary detectable probes that hybridize to the first encoding probe and/or the one or more second encoding probes.

In some embodiments, the RCA product of the first encoding probe is a first RCA product. In some embodiments, the circular or circularized one or more second encoding probes are used as template to generate one or more second RCA products, which may be used to generate and detect a signal. The signal associated with the first RCA product and the signal associated with the one or more second RCA products may be detected simultaneously or sequentially in either order. The first RCA product and/or the one or more second RCA products may be contacted with one or more secondary detectable probes that each directly or indirectly bind to an RCA product. Independently, each secondary detectable probe may directly or indirectly bind (e.g. hybridize) to a complementary barcode sequence in the first RCA product or the one or more second RCA products. In some embodiments, the complementary barcode sequence in the first RCA product or the one or more second RCA products is the complement of a amplifiable barcode sequence in the first encoding probe or the one or more second encoding probes. In some embodiments, independently, each secondary detectable probe is fluorescently labeled or hybridizes to a fluorescently labeled probe. The secondary detectable probes may be hybridized, in sequential cycles, to the complementary barcode sequences in the first RCA product or the one or more second RCA products. In some embodiments, a temporal order of signals in the sequential cycles associated with the secondary detectable probes corresponds to the complementary barcode sequence which corresponds to the target nucleic acid or a portion thereof.

In some embodiments, the method comprises using signals associated with the first RCA product to select a first number of second encoding probes for RCA, or using signals associated with the RCA products of the first number of second encoding probes to select a second number of second encoding probes for RCA. In some embodiments, two or more of the second encoding probes comprise different primer binding sequences for RCA. The method may comprise using signals associated with the one or more primary detectable probes to select a duration of RCA to generate the RCA product of the first encoding probe. The method may comprise using signals associated with the RCA product of the first encoding probe to select a number of second encoding probes for RCA and/or to select a duration of RCA to generate RCA product(s) of the one or more second encoding probes.

In some embodiments, provided herein is a method for analyzing a biological sample. In some embodiments, the method comprises: a) contacting the biological sample with a plurality of encoding probes. In some embodiments, the plurality of encoding probes comprises a first encoding probe, an alternative first encoding probe, and one or more second encoding probes. In some embodiments, the first/alternative first encoding probe and each second encoding probe are capable of hybridizing to a first target sequence and a second target sequence, respectively, in a target nucleic acid in the biological sample. In some embodiments, the first encoding probe and the alternative first encoding probe are circularizable and each comprises an interrogatory region for interrogating a region of interest in the first target sequence. In some embodiments, the interrogatory region of the first encoding probe is complementary to the region of interest and the interrogatory region of the alternative first encoding probe is not complementary to the region of interest. In some embodiments, each second encoding probe is circular or circularizable. In some embodiments, the method further comprises: b) circularizing the first encoding probe hybridized to the region of interest to generate a circularized first encoding probe using the first target sequence as template, wherein the alternative first encoding probe is not sufficiently complementary to the region of interest to allow circularization of the alternative first encoding probe. In some embodiments, the method further comprises: c) contacting the biological sample with one or more primary detectable probes that hybridize to the first/alternative first encoding probe and/or the second encoding probe. In some embodiments, the method further comprises: d) detecting a signal associated with the one or more primary detectable probes. In some embodiments, the method further comprises: e) generating a rolling circle amplification (RCA) product of the circularized first encoding probe. In some embodiments, the method further comprises: f) detecting a signal associated with the RCA product. In some embodiments, circularizing the first encoding probe hybridized to the region of interest to generate a circularized first encoding probe is performed before contacting the sample with primary detectable probes.

In some embodiments, provided herein is a method for analyzing a biological sample. In some embodiments, the method comprises: a) contacting the biological sample with a plurality of encoding probes, wherein: the plurality of encoding probes comprises a first encoding probe and one or more second encoding probes, the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, the region of interest is complementary to the interrogatory region, each second encoding probe is circular or circularizable, and the biological sample comprises a counterpart target nucleic acid comprising (i) a counterpart first target sequence having the same sequence as the first target sequence except that the region of the counterpart first target sequence corresponding to the region of interest is not complementary to the interrogatory region in the first encoding probe, and (ii) the second target sequence. In some embodiments, the method further comprises: b) circularizing the first encoding probe to generate a circularized first encoding probe using the first target sequence as template, and not using the counterpart first target sequence as template. In some embodiments, the method further comprises: c) contacting the biological sample with one or more primary detectable probes that hybridize to the first encoding probe and/or the one or more second encoding probe. In some embodiments, the method further comprises: d) detecting a signal associated with the one or more primary detectable probes. In some embodiments, the method further comprises: e) generating a rolling circle amplification (RCA) product of the circularized first encoding probe. In some embodiments, the method further comprises: f) detecting a signal associated with the RCA product.

In some embodiments, provided herein is a method for analyzing a biological sample. In some embodiments, the method comprises: a) contacting the biological sample with a plurality of encoding probes, wherein: the plurality of encoding probes comprises a first encoding probe, an alternative first encoding probe, and one or more second encoding probes, the first encoding probe and each second encoding probe are capable of hybridizing to a first target sequence and a second target sequence, respectively, in a target nucleic acid in the biological sample, the alternative first encoding probe and each second encoding probe are capable of hybridizing to a counterpart first target sequence and the second target sequence, respectively, in a counterpart target nucleic acid in the biological sample, the first encoding probe is circularizable and comprises an interrogatory region for interrogating a region of interest in the first target sequence, the alternative first encoding probe is circularizable and comprises an alternative interrogatory region for interrogating a counterpart region of interest in the counterpart first target sequence, and each second encoding probe is circular or circularizable. In some embodiments, the method further comprises: b) circularizing the first encoding probe to generate a circularized first encoding probe using the first target sequence as template, and circularizing the alternative first encoding probe to generate a circularized alternative first encoding probe using the counterpart first target sequence as template. In some embodiments, the method further comprises: c) contacting the biological sample with one or more primary detectable probes that hybridize to the first encoding probe, the alternative first encoding probe, and/or the second encoding probe. In some embodiments, the method further comprises: d) detecting a signal associated with the one or more primary detectable probes. In some embodiments, the method further comprises: e) generating a rolling circle amplification (RCA) product of the circularized first encoding probe and an RCA product of the circularized alternative first encoding probe. In some embodiments, the method further comprises: f) detecting a signal associated with the RCA product of the circularized first encoding probe and/or a signal associated with the RCA product of the circularized alternative first encoding probe.

The counterpart first target sequence may have the same sequence the first target sequence except that the counterpart region of interest is different than the region of interest. The region of interest and the counterpart region of interest may be different alleles of the group consisting of a single-nucleotide polymorphism (SNP), a single-nucleotide variant (SNV), a single-nucleotide substitution, a point mutation, a single-nucleotide insertion, and a single-nucleotide deletion. In some embodiments, the first encoding probe is not circularized using the counterpart first target sequence as template, and/or the alternative first encoding probe is not circularized using the first target sequence as template. The first encoding probe and the alternative first encoding probe may comprise different barcode regions. In some embodiments, signals associated with the RCA product of the circularized first encoding probe and signals associated with the RCA product of the circularized alternative first encoding probe are detected in different locations of the biological sample.

In some aspects, provided herein is a method for analyzing a biological sample. In some embodiments, the method comprises contacting the biological sample with a plurality of encoding probes or probe sets each capable of hybridizing to a target sequence in a target nucleic acid in the biological sample. The method can comprise ligating the ends of the encoding probes or probes sets to form a ligation product. The method can further comprise contacting the biological sample with a plurality of detectable probes that hybridize to the plurality of encoding probes or probe sets. In some embodiments, each encoding probe or probe set forms an amplification complex with two or more detectable probes. In some embodiments, the method further comprises detecting a signal associated with the two or more detectable probes. In some embodiments, an encoding probe set comprises a first probe and a second probe. The first and/or second probe may comprise an overhang that is for binding (e.g. hybridizing) directly or indirectly to the detectable probes, and that does not hybridize to the target nucleic acid. Ligation of the first and second probes may comprise two ligations to join the first and second probes at both ends. Each of the detectable probes may comprise a region for directly or indirectly binding (e.g. hybridizing) two or more fluorescently labeled probes. The first encoding probe may be circularizable, and may be circularized with or without gap filling prior to ligation. In some embodiments, upon hybridization to the target nucleic acid, a flap region on the 5′ and/or 3′ end of the first encoding probe is formed, and the flap region or a portion thereof is cleaved prior to the ligation. In some embodiments, upon hybridization to the target nucleic acid, a flap region on the 5′ and/or 3′ end of the first encoding probe is not formed.

In some embodiments, the methods provided herein are performed in situ in the biological sample. In some embodiments, one or more of the signals are detected in situ in the biological sample.

In some embodiments, provided herein is a kit for analyzing a biological sample, comprising: a) a plurality of encoding probes comprising a first encoding probe and one or more second encoding probes, wherein: the first encoding probe and each second encoding probe are capable of hybridizing to a first target sequence and a second target sequence, respectively, in a target nucleic acid in the biological sample, the first encoding probe is circularizable and comprises an interrogatory region for interrogating a region of interest in the first target sequence, and each second encoding probe is circular or circularizable; and b) one or more primary detectable probes that hybridize to the first encoding probe and/or the second encoding probe. The kit may further comprise a ligase for circularizing the first encoding probe to generate a circularized first encoding probe, and optionally for circularizing one or more second encoding probes to generate one or more circularized second encoding probes. The kit may further comprise one or more secondary detectable probes that directly or indirectly bind (e.g. hybridize) to an RCA product of the first encoding probe or an RCA product of one or more second encoding probes. In some embodiments, the one or more second encoding probes in the kit comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more second encoding probes that each hybridize to a different second target sequence in the target nucleic acid.

### II. Samples, Analytes, and Target Sequences

A. Samples

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., one or more probes such as a circular or circularizable probe (e.g. a padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe (e.g. padlock probe).

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, for example as described in Chen et al., Science 347(6221):543-548, 2015, the content of which is herein incorporated by reference in its entirety.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9×its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the amount of probes in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Any suitable non-chemical permeabilization methods can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each analyte (e.g. RNA or cDNA) are used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and/or alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected. In some embodiments, the analyte is a nucleic acid, such as a target nucleic acid described herein.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a circularizable probe such as a padlock probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly binds (e.g. hybridizes) to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example by using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (e.g. coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Target Sequences

In some embodiments, provided herein are methods and compositions for analyzing one or more analytes, products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

A target sequence for a probe (e.g., encoding probe) disclosed herein may be comprised in any analyte disclose herein, including an endogenous analyte (e.g., a viral or cellular nucleic acid), a labeling agent, or a product of an endogenous analyte and/or a labeling agent. In some embodiments, a target sequence for a probe (e.g., encoding probe) disclosed herein comprises one or more ribonucleotides.

### III. Nucleic Acid Probes

Disclosed herein in some aspects are nucleic acid probes and/or probe sets (e.g., encoding probes and/or probe sets) that are introduced into a cell or used to otherwise contact a biological sample such as a tissue sample. The probes (e.g., the encoding probes disclosed herein and/or any detectable probe disclosed herein, e.g., for FISH (such as single molecule FISH (smFISH)) and/or RCA-based detection) may comprise any of a variety of entities that can hybridize to a nucleic acid, typically by Watson-Crick base pairing, such as DNA, RNA, LNA, PNA, etc. The nucleic acid probe may comprise a targeting sequence that is able to directly or indirectly bind to at least a portion of a target nucleic acid. The nucleic acid probe may be able to bind to a specific target nucleic acid (e.g., an mRNA, or other nucleic acids disclosed herein). In some embodiments, the nucleic acid probes may be detected using a detectable label, and/or by using secondary nucleic acid probes able to bind to the nucleic acid probes. In some embodiments, nucleic acid probes and/or probe sets (e.g., encoding probes and/or probe sets) can be used to generate smFISH and/or RCA signals associated with a target nucleic acid or portion thereof.

In some embodiments, an smFISH signal is a signal generated using a method comprising single molecule FISH (smFISH), which does not comprise an amplification step. In some embodiments, an smFISH signal is a signal generated using a method that does not comprise enzymatic extension. In some embodiments, an RCA signal is a signal generated by detecting an RCA product.

In some embodiments, the nucleic acid probes (e.g., primary probes and/or secondary probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

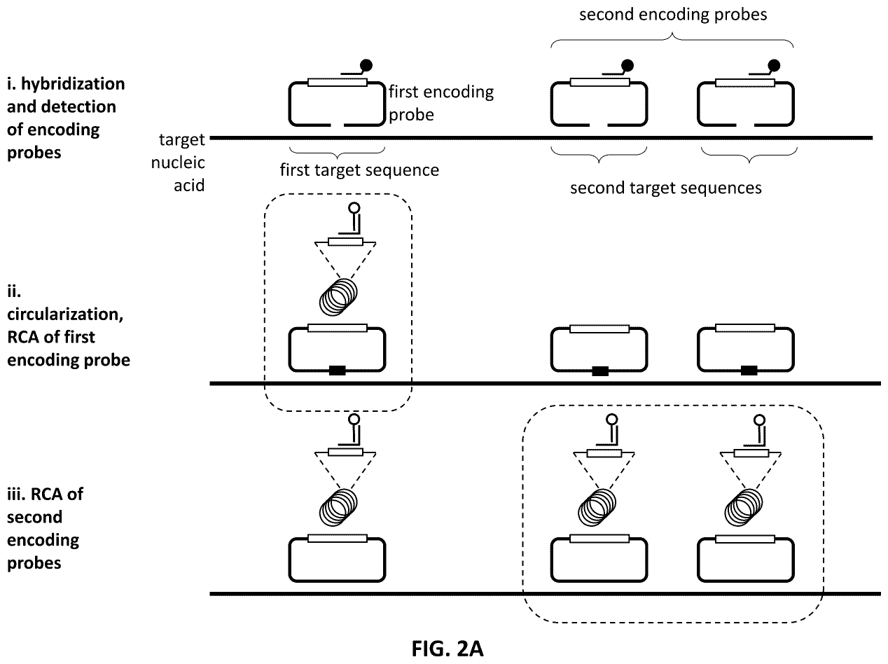

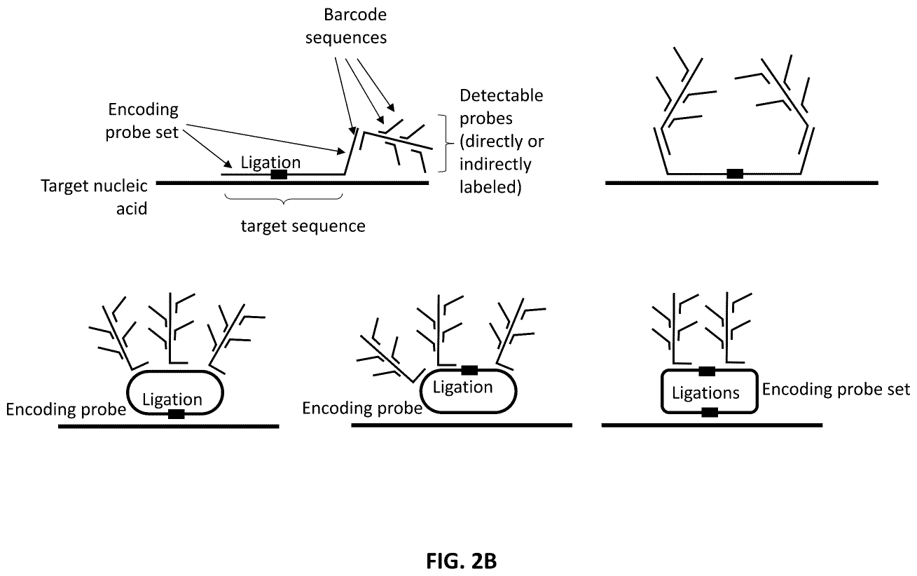

In some aspects, the methods disclosed herein comprise a plurality of primary probes (e.g., encoding probes) that hybridize to the target nucleic acid. In some embodiments, the nucleic acid probes are detected using secondary nucleic acid probes and detectable probes in a hybridization complex that generates an amplified signal (e.g., as described in Section V), which allows fewer primary probes to be needed to generate a sufficient detectable signal. In some embodiments, amplifiers (e.g. secondary or higher order nucleic acid probes) are hybridized and/or directly or indirectly bound (via one or more oligonucleotides) to a target nucleic acid (e.g., as shown in FIG. 2B). In some aspects, the biological sample may be contacted with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more encoding probes each hybridizing to a different target sequence in the target nucleic acid. For example, fewer than about 30, at least about 25, at least about 20, at least about 15, at least about 10, or at least about 5 encoding probes can be needed to hybridize to each target nucleic acid to generate a sufficient detectable signal. In some instances, no more than 5 encoding probes are hybridized to each target nucleic acid (e.g., which may be tiled across the nucleic acid molecule).

In some embodiments, more than one type of primary nucleic acid probes (e.g., encoding probes) may be contacted with a sample, e.g., simultaneously or sequentially in any suitable order, such as in sequential probe hybridization/unhybridization cycles. In some instances, the sample is contacted with linear primary probes (e.g., a first and second probe). In some instances, the sample is contact with circularizable primary probes. In some embodiments, the sample is contacted with both linear and circularizable primary probes (simultaneously or sequentially in any suitable order). In some embodiments, more than one type of secondary nucleic acid probes may be contacted with a sample, e.g., simultaneously or sequentially in any suitable order, such as in sequential probe hybridization/unhybridization cycles. In some embodiments, the secondary probes may comprise probes that bind to a product of a primary probe targeting an analyte. In some embodiments, more than one type of higher order nucleic acid probes may be contacted with a sample, e.g., simultaneously or sequentially in any suitable order, such as in sequential probe hybridization/unhybridization cycles. In some embodiments, more than one type of detectably labeled nucleic acid probes (e.g., one or more primary detectable probes for smFISH readout and/or one or more secondary detectable probes for RCA readout) may be contacted with a sample, e.g., simultaneously or sequentially in any suitable order, such as in sequential probe hybridization/unhybridization cycles. In some embodiments, the detectably labeled nucleic acid probes can be used for both smFISH readout and for RCA readout. In some embodiments, the detectably labeled probes (e.g., the one or more primary detectable probes for smFISH readout and/or the one or more secondary detectable probes for RCA readout as described herein) may comprise probes that bind to one or more primary probes (e.g., encoding probes), one or more secondary probes, one or more higher order probes, one or more intermediate probes between a primary/secondary/higher order probes, and/or one or more detectably or non-detectably labeled probes (e.g., as in the case of a hybridization chain reaction (HCR), a branched DNA reaction (bDNA), or the like). In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., primary, secondary, higher order probes, and/or detectably labeled probes) can be contacted with a sample, e.g., simultaneously or sequentially in any suitable order. Between any of the probe contacting steps disclosed herein, the method may comprise one or more intervening reactions and/or processing steps, such as wash steps (e.g. stringent wash), modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The target-binding sequence (sometimes also referred to as the targeting region/sequence, the recognition region/sequence, or the hybridization region/sequence) of a probe or probe set may be positioned anywhere within the probe. For instance, the target-binding sequence of a primary probe (e.g., the hybridization region of an encoding probe) that binds to a target nucleic acid can be 5′ or 3′ to any barcode sequence in the primary probe. Likewise, the target-binding sequence of a secondary probe (which binds to a primary probe or complement or product thereof) can be 5′ or 3′ to any barcode sequence in the secondary probe. In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The target-binding sequence of a primary nucleic acid probe or probe set may be determined with reference to a target nucleic acid (e.g., a cellular RNA or a reporter oligonucleotide of a labeling agent for a cellular analyte) that is present or suspected of being present in a sample. In some embodiments, more than one target-binding sequence can be used to identify a particular analyte comprising or associated with a target nucleic acid. The more than one target-binding sequence can be in the same probe or in different probes (e.g., two or more probes to be ligated as shown in FIG. 2B). For instance, multiple probes can be used, sequentially and/or simultaneously, that can bind to (e.g., hybridize to) different regions of the same target nucleic acid. In other examples, a probe may comprise target-binding sequences that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

After contacting the nucleic acid probes with a sample, the probes may be directly detected by determining detectable labels (if present), and/or detected by using one or more other probes that bind directly or indirectly to the probes or products thereof. The one or more other probes may comprise a detectable label. For instance, a primary nucleic acid probe can bind to a target nucleic acid in the sample, and a secondary nucleic acid probe can be introduced to bind to the primary nucleic acid probe, where the secondary nucleic acid probe or a product thereof can then be detected using detectable probes (e.g., detectably labeled probes). Higher order probes that directly or indirectly bind to the secondary nucleic acid probe or product thereof may also be used, and the higher order probes or products thereof can then be detected using detectably labeled probes.

In some instances, a secondary nucleic acid probe binds to a primary nucleic acid probe (e.g., encoding probe) directly hybridized to the target nucleic acid. A secondary nucleic acid probe (e.g., a primary detectable probe or a secondary detectable probe disclosed herein) may contain a recognition sequence able to bind to or hybridize with a primary nucleic acid probe (e.g., a circularizable encoding probe disclosed herein) or a product thereof (e.g., a hybridization product or an RCA product), e.g., at a barcode sequence or portion(s) thereof of the primary nucleic acid probe or product thereof. In some embodiments, a secondary nucleic acid probe may bind to a combination of barcode sequences (which may be continuous or spaced from one another) in a primary nucleic acid probe, a product thereof, or a combination of primary nucleic acid probes. In some embodiments, the binding is specific, or the binding may be such that a recognition sequence preferentially binds to or hybridizes with only one of the barcode sequences or complements thereof that are present. The secondary nucleic acid probe may also contain one or more detectable labels. If more than one secondary nucleic acid probe is used, the detectable labels may be the same or different.

The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In some embodiments, the recognition sequence is of the same length as a barcode sequence or complement thereof of a primary nucleic acid probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, a nucleic acid probe, such as a primary or a secondary nucleic acid probe, may also comprise 1, 2, 3, 4, 5, 6, 7, 8, 9, 10, 11, 12, 13, 14, 15 or more, 20 or more, 32 or more, 40 or more, or 50 or more barcode sequences. As an illustrative example, a first probe may contain a first target-binding sequence, a first barcode sequence, and a second barcode sequence, while a second, different probe may contain a second target-binding sequence (that is different from the first target-binding sequence in the first probe), the same first barcode sequence as in the first probe, but a third barcode sequence instead of the second barcode sequence. Such probes may thereby be distinguished by determining the various barcode sequence combinations present or associated with a given probe at a given location in a sample.

In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode may be a barcode region. In some embodiments, a barcode comprises two or more sub-barcodes that together function as a single barcode. For example, a polynucleotide barcode can include two or more polynucleotide sequences (e.g., sub-barcodes) that are separated by one or more non-barcode sequences.

In some embodiments, barcodes or complements thereof (e.g., barcode sequences or complements thereof comprised by the probes disclosed herein or products thereof) can be analyzed (e.g., detected or sequenced) using any suitable method or technique, including those described herein, such as sequencing by synthesis (SBS), sequencing by ligation (SBL), or sequencing by hybridization (SBH). In some instances, barcoding schemes and/or barcode detection schemes as described in RNA sequential probing of targets (RNA SPOTs), single-molecule fluorescent in situ hybridization (smFISH), multiplexed error-robust fluorescence in situ hybridization (MERFISH) or sequential fluorescence in situ hybridization (seqFISH+) can be used. In any of the preceding implementations, the methods provided herein can include analyzing the barcodes by sequential hybridization and detection with a plurality of labeled probes (e.g., detection probes (e.g., detection oligos) or barcode probes, such as primary detectable probes or secondary detectable probes described herein). In some instances, the barcode detection steps can be performed as described in hybridization-based in situ sequencing (HybISS). In some instances, probes can be detected and analyzed (e.g., detected or sequenced) as performed in fluorescent in situ sequencing (FISSEQ), or as performed in the detection steps of the spatially-resolved transcript amplicon readout mapping (STARmap) method. In some instances, signals associated with an analyte can be detected as performed in sequential fluorescent in situ hybridization (seqFISH).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCA products are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub. 20210164039, which are each hereby incorporated by reference in their entirety.

In some embodiments, the nucleic acid probes disclosed herein may be made using only 2 or only 3 of the 4 bases, such as leaving out all the “G”s and/or leaving out all of the “C”s within the probe. Sequences lacking either “G”s or “C”s may form very little secondary structure, and can contribute to more uniform, faster hybridization in certain embodiments.

In some embodiments, a nucleic acid probe disclosed herein may comprise a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe disclosed herein lacks a detectable label. While a detectable label may be incorporated into an amplification product of a probe, such as via incorporation of a modified nucleotide into an RCA product of a circularized probe, the amplification product itself in some embodiments is not detectably labeled. In some embodiments, a probe that binds to the primary nucleic acid probe or a product thereof (e.g., a secondary nucleic acid probe that binds to a barcode sequence or complement thereof in the primary nucleic acid probe or product thereof) comprises a detectable label and may be used to detect the primary nucleic acid probe or product thereof. In some embodiments, a secondary nucleic acid probe disclosed herein lacks a detectable label, and a detectably labeled probe that binds to the secondary nucleic acid probe or a product thereof (e.g., at a barcode sequence or complement thereof in the secondary nucleic acid probe or product thereof) can be used to detect the secondary nucleic acid probe or product thereof. In some embodiments, signals associated with the detectably labeled probes can be used to detect one or more barcode sequences in the secondary probe and/or one or more barcode sequences in the primary probe, e.g., by using sequential hybridization of detectably labeled probes, sequencing-by-ligation, and/or sequencing-by-hybridization. In some embodiments, the barcode sequences (e.g., in the secondary probe and/or in the primary probe) are used to combinatorially encode a plurality of analytes of interest, such as target nucleic acids. As such, signals associated with the detectably labeled probes at particular locations in a biological sample can be used to generate distinct signal signatures that each corresponds to an analyte in the sample, thereby identifying the analytes at the particular locations, e.g., for in situ spatial analysis of the sample.

In some embodiments, a nucleic acid probe herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order.

In some aspects, analytes can be targeted by primary probes or probe sets, which include one or more barcode sequences (e.g., sequences that can be detected or otherwise “read”) that are separate from a sequence in a primary probe that directly or indirectly binds the targeted analyte. In some embodiments, the primary probes are in turn targeted by secondary probes, which can also include one or more barcode sequences that are separate from a recognition sequence in a secondary probe that directly or indirectly binds a primary probe or a product thereof. In some embodiments, a secondary probe may bind to a barcode sequence in the primary probe. In some aspects, tertiary probes and optionally even higher order probes may be used to target the secondary probes, e.g., at a barcode sequence or complement thereof in a secondary probe or product thereof. In some embodiments, the probes, such as primary, secondary, or tertiary probes and/or even higher order probes may comprise one or more barcode sequences and/or one or more detectable labels. For example, in some embodiments, a tertiary probe is a detectably labeled probe that hybridizes to a barcode sequence (or complement thereof) of a secondary probe (or product thereof). In some embodiments, through the detection of signals associated with detectably labeled probes in a sample, the location of one or more analytes in the sample and the identity of the analyte(s) can be determined. In some embodiments, the presence/absence, absolute or relative abundance, an amount, a level, a concentration, an activity, and/or a relation with another analyte of a particular analyte can be analyzed in situ in the sample.

In some embodiments, provided herein are probes, probe sets, and assay methods to couple target nucleic acid detection, signal amplification (e.g., through nucleic acid amplification such as RCA, and/or hybridization of a plurality of detectably labeled probes, such as in hybridization chain reactions and the like), and decoding of the barcodes.

In some aspects, a primary probe or probe set, a secondary probe, and/or a higher order probe (e.g., encoding probe, detectably labeled nucleic acid probes such as primary detectable probes for smFISH readout and/or secondary detectable probes for RCA readout) can be selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes.

Specific probe designs can vary depending on the application. For instance, a primary probe, a secondary probe, and/or a higher order probe disclosed herein can comprise a circularizable probe that does not require gap filling to circularize upon hybridization to a template (e.g., a target nucleic acid and/or a probe such as a splint), a gapped circularizable probe (e.g., one that requires gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), or any suitable combination thereof. In some embodiments, a primary probe, a secondary probe, and/or a higher order probe disclosed herein can comprise a probe that is ligated to itself or another probe using DNA-templated and/or RNA-templated ligation. In some embodiments, a primary probe, a secondary probe, and/or a higher order probe disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template. In some embodiments, a primary probe, a secondary probe, and/or a higher order probe disclosed herein can comprise a padlock-like probe or probe set, such as one described in US 2019/0055594, US 2021/0164039, US 2016/0108458, or US 2020/0224243, each of which is incorporated herein by reference in its entirety. Any suitable combination of the probe designs described herein can be used.

In some embodiments, a primary probe (e.g., encoding probe), a secondary probe, and/or a higher order probe disclosed herein can comprise two or more parts. In some cases, a probe can comprise one or more features of and/or be modified based on: a split FISH probe or probe set described, for example, in WO 2021/167526 A1 or Goh et al., “Highly specific multiplexed RNA imaging in tissues with split-FISH,” Nat Methods 17(7):689-693 (2020), which are each incorporated herein by reference in their entireties; a Z-probe or probe set, such as one described in U.S. Pat. No. 7,709,198 B2, U.S. Pat. No. 8,604,182 B2, U.S. Pat. No. 8,951,726 B2, U.S. Pat. No. 8,658,361 B2, or Tripathi et al., “Z Probe, An Efficient Tool for Characterizing Long Non-Coding RNA in FFPE Tissues,” Noncoding RNA 4(3):20 (2018), which are each incorporated herein by reference in their entireties; an HCR initiator or amplifier, such as one described in U.S. Pat. No. 7,632,641 B2, US 2017/0009278 A1, U.S. Pat. No. 10,450,599 B2, Dirks and Pierce, “Triggered amplification by hybridization chain reaction,” PNAS 101(43):15275-15278 (2004), Chemeris et al., “Real-time hybridization chain reaction,” Dokl. Biochem 419:53-55 (2008), Niu et al., “Fluorescence detection for DNA using hybridization chain reaction with enzyme-amplification,” Chem Commun (Camb) 46(18):3089-91 (2010), Choi et al., “Programmable in situ amplification for multiplexed imaging of mRNA expression,” Nat Biotechnol 28(11):1208-12 (2010), Song et al., “Hybridization chain reaction-based aptameric system for the highly selective and sensitive detection of protein,” Analyst 137(6):1396-401 (2012), Choi et al., “Third-generation in situ hybridization chain reaction: multiplexed, quantitative, sensitive, versatile, robust,” Development 145(12): dev165753 (2018), or Tsuneoka and Funato, “Modified in situ Hybridization Chain Reaction Using Short Hairpin DNAs,” Front Mol Neurosci 13:75 (2020), which are each incorporated herein by reference in their entireties; a PLAYR probe or probe set, such as one described in US 2016/0108458 A1 or Frei et al., “Highly multiplexed simultaneous detection of RNAs and proteins in single cells,” Nat Methods 13(3):269-75 (2016), which are each incorporated herein by reference in their entireties; a PLISH probe or probe set, such as one described in US 2020/0224243 A1 or Nagendran et al., “Automated cell-type classification in intact tissues by single-cell molecular profiling,” eLife 7:e30510 (2018), which are each incorporated herein by reference in their entireties; a RollFISH probe or probe set such as one described in Wu et al., “RollFISH achieves robust quantification of single-molecule RNA biomarkers in paraffin-embedded tumor tissue samples,” Commun Biol 1, 209 (2018), which is hereby incorporated by reference in its entirety; a MERFISH probe or probe set, such as one described in US 2022/0064697 A1 or Chen et al., “Spatially resolved, highly multiplexed RNA profiling in single cells,” Science 348(6233):aaa6090 (2015), which are each incorporated herein by reference in their entireties; or a primer exchange reaction (PER) probe or probe set, such as one described in US 2019/0106733 A1, which is hereby incorporated by reference in its entirety.

In some embodiments, an encoding probe in the plurality of encoding probes comprises one or more features and/or is modified to allow for generation and detection of a first signal that does not comprise a nucleic acid amplification step (e.g., the first signal can be an smFISH signal). In some embodiments, the plurality of encoding probes comprises probes that directly hybridize to multiple regions (e.g., sequences) of the same transcript. In some embodiments, the encoding probes comprise one or more features and/or are modified to allow for generation and detection of a second signal that comprises an amplification step (e.g., extension and/or amplification catalyzed by a polymerase).

Any suitable circularizable probe or probe set, or indeed more generally circularizable reporter molecules, may be used to generate the RCA template which is used to generate the RCA product. In some embodiments, a circularizable probe or reporter is in the form of a linear molecule having ligatable ends which may be ligated to one another form a circularized molecule (e.g. to serve as the RCA template). The ends may be ligated together directly or indirectly, e.g. to each other, or to the respective ends of an intervening (“gap”) oligonucleotide or to an extended 3′ end of the circularizable probe. A circularizable probe may also be provided in two or more parts, namely two or more molecules (e.g. oligonucleotides) which may be ligated together to form a circle. Ligation may be templated using a ligation template. The circularizable probe (or template part or portion) may comprise at its respective 3′ and 5′ ends regions of complementarity to corresponding cognate complementary regions (or binding sites) in the ligation template, which may be adjacent to where the ends are directly ligated to each other, or non-adjacent, with an intervening “gap” sequence, where indirect ligation is to take place.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

In some aspects, the methods provided herein comprise performing rolling circle amplification of a circular probe or a circularized probe generated from a circularizable probe or probe set (e.g. RCA of a circular or circularized encoding probe or probe set).

In some embodiments, a probe disclosed herein can comprise a 5′ flap which may be recognized by a structure-specific cleavage enzyme, e.g. an enzyme capable of recognizing the junction between a single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to a target, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiment, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/0224244, each of which are herein incorporated by reference in their entireties, and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 (the content of which is herein incorporated by reference in its entirety) for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

### IV. Probe Based Assays and Generation of Multiple Signals in a Sample

In some aspects, provided herein are methods comprising contacting a biological sample with a plurality of encoding probes and detecting signals associated with the encoding probes. In some embodiments, each of the encoding probes comprises a hybridization region which is complementary to a target sequence in the biological sample (e.g. a target sequence in a target nucleic acid). In some instances, the plurality of encoding probes comprises a first encoding probe and one or more second encoding probes. In some embodiments, the first encoding probe comprises a first hybridization region which is complementary to a first target sequence in the biological sample (e.g. in a target nucleic acid) and one or more second encoding probes comprise one or more second hybridization regions which are complementary to one or more second target sequences in the same biological sample (e.g. in the same target nucleic acid). In some aspects, the first and second target sequences are different. In some aspects, the first and second target sequences are in the same analytes of interest. In some instances, an assay can be performed comprising generating and detecting signals associated with a target nucleic acid using one or more encoding probes and one or more signal generation methods (e.g., combinations of smFISH signals, non-enzymatically amplified signals (e.g., assembly of branched structures for signal amplification), and/or enzymatically amplified signals (e.g., generating an RCA product)). In some embodiments, a target nucleic acid is contacted with a plurality of encoding probes or probe sets that hybridize to various sequences along the target nucleic acid and a subset of the plurality of encoding probe or probe sets is used to generate an amplified signal.

In some embodiments, one or more barcode regions are located at one or more locations (e.g., 5′ end, 3′ end, and/or between the 5′ and 3′ ends) on the first encoding probe and one or more second encoding probe. In some embodiments, the one or more barcode regions are target-specific (e.g., a sequence of the barcode region can uniquely correspond to the target nucleic acid or subsequence thereof targeted by the first encoding probe and/or one or more second encoding probes). In some embodiments, the one or more barcode regions per se are not target-specific, but a combination of the one or more barcode regions with other barcode(s) or barcode region(s) is target-specific, and the combination can be used to identify the target nucleic acid or subsequence thereof. In certain embodiments, a primary detectable probe hybridizes to, or binds directly or indirectly to, one or more barcode regions comprised by an encoding probe. In certain embodiments, a secondary detectable probe hybridizes to, or binds directly or indirectly to, one or more barcode regions or complements thereof, or to an RCA product comprising one or more barcode regions or complements thereof (e.g. an RCA product of an encoding probe).

In certain embodiments, a primary detectable probe can hybridize to one or more barcode regions before the encoding probe contacts the biological sample. In certain embodiments, the primary detectable probe can hybridize to the one or more barcode regions after the circular or circularizable probe contacts the biological sample. In certain embodiments, the primary detectable probe can hybridize to one or more barcode regions before the secondary detectable probe hybridizes to the one or more barcode regions or RCA products comprising complements of the one or more barcode regions. In certain embodiments, the primary detectable probe can hybridize to one or more barcode regions after the secondary detectable probe hybridizes to one or more barcode regions or RCA products comprising complements of a barcode region. In certain embodiments, a primary detectable probe can hybridize to one or more barcode regions before circularizing the first encoding probe and/or generating a rolling circle amplification (RCA) product of the circularized first encoding probe. In certain embodiments, a primary detectable probe can hybridize to one or more barcode regions after circularizing the first encoding probe and/or generating a rolling circle amplification (RCA) product of the circularized first encoding probe.

In certain embodiments, both the signal associated with the primary detectable probe and the signal associated with the secondary detectable probe are detected simultaneously or in any suitable order.

In certain embodiments, the signal associated with the primary detectable probe is detected after the plurality of encoding probes contacts the biological sample. In certain embodiments, the signal associated with the primary detectable probe is detected before the secondary detectable probe hybridizes to the one or more barcode regions or RCA products comprising complements of one or more barcode regions. In certain embodiments, the signal associated with the primary detectable probe is detected after the secondary detectable probe hybridizes to one or more barcode regions or RCA products comprising complements of one or more barcode regions. In certain embodiments, the signal associated with the primary detectable probe is detected before the signal associated with the secondary detectable probe is detected. In certain embodiments, the signal associated with the primary detectable probe is detected after the signal associated with the secondary detectable probe is detected.

In certain embodiments, the first target sequence can be a continuous sequence or a split sequence comprising two or more subsequences in the same nucleotide molecule or in different nucleotide molecules. In certain embodiments, the first target sequence can be a continuous sequence in a genomic DNA, a cDNA, an mRNA, a viral RNA, or a non-coding RNA. In certain embodiments, the two or more subsequences are in multiple oligonucleotide probes that hybridize to a genomic DNA, a cDNA, an mRNA, a viral RNA, or a non-coding RNA.

In some embodiments, the first target sequence comprises a region of interest. In some embodiments, the region of interest is a single nucleotide of interest or a dinucleotide of interest. In some embodiments, the region of interest is selected from the group consisting of a single-nucleotide polymorphism (SNP), a single-nucleotide variant (SNV), a single-nucleotide substitution, a point mutation, a single-nucleotide insertion, or a single-nucleotide deletion. In some embodiments, the region of interest comprises a splice junction.

In some embodiments, the encoding probes can be circularized. In some embodiments, a circularized encoding probe is used as a template to generate an RCA product. In some embodiments, one or more encoding probes is not amplified or processed by a polymerase for RCA. In certain embodiments, one or more second encoding probes comprises a modification to protect from extension or otherwise processing (e.g., cleavage) by the polymerase for RCA, whereas the polymerase can amplify and/or otherwise process the first encoding probe. In some embodiments, the modification of the encoding probe confers resistance to 5′->3′ polymerase activity and/or a 3′->5′ exonuclease activity of the polymerase. In some embodiments, the polymerase for RCA is Phi29.

In some embodiments, an encoding probe is provided as a single probe molecule (e.g. a padlock probe). In certain embodiments, an encoding probe comprises two or more probe molecules which may but do not need to be connected (e.g., via enzymatic or chemical ligation) to form a single probe molecule. In certain embodiments, an encoding probe comprises two or more probe molecules which are configured to be connected via one or more ligations (e.g., via enzymatic or chemical ligation) to form a single probe molecule. In certain embodiments, an encoding probe comprises two probe molecules each comprising a sequence that hybridizes to a subsequence of the first target sequence, and the two probe molecules each further comprises a sequence that hybridizes to a subsequence of a splint (e.g., a primary detectable probe can serve as the splint). In some embodiments, the splint (e.g., a primary detectable probe) hybridizes to one or more barcode regions. In some embodiments, the splint (e.g., a primary detectable probe) hybridizes to a barcode region which is a split barcode region. The two or more probe molecules of an encoding probe can but do not need to be connected (e.g., via enzymatic or chemical ligation) using a target sequence as a template, with or without gap filling. Likewise, the two or more probe molecules of an encoding probe set can but do not need to be connected (e.g., via enzymatic or chemical ligation) using a splint (e.g., a primary detectable probe) as a template, with or without gap filling.

In certain embodiments, upon contacting the biological sample with a plurality of primary detectable probes, the primary detectable probes bind to a sequence in one or more barcode regions. In certain embodiments, the primary detectable probes are cleavable or degradable. In certain embodiments, the contacting of the biological sample by the primary detectable probes and the detection of a signal associated with the primary detectable probe are performed sequentially, e.g., in sequential detectable probe hybridization cycles, wherein in each cycle, the encoding probes hybridized to target sequences in the biological sample are contacted with one or more primary detectable probes.

In certain embodiments, one or more encoding probes is not circularized upon hybridization to a target sequence. In certain embodiments, an encoding probe is circularized upon hybridization to a target sequence. In certain embodiments, one or more circularized encoding probes is not used as a template to generate an RCA product for detection. In certain embodiments, one or more circularized encoding probes is used as a template to generate an RCA product for detection.

In certain embodiments, the primary detectable probe comprises a detectable label. In certain embodiments, the primary detectable probe comprises a fluorescent moiety. In certain embodiments, a signal associated with the fluorescent moiety can be extinguished by unhybridizing the primary detectable probe from an encoding probe; enzymatically cleaving the fluorescent moiety from the primary detectable probe; masking, removing or modifying the fluorescent moiety; cleaving a linker linking the fluorescent moiety to the primary detectable probe; chemically or photochemically modifying the fluorescent moiety; bleaching the fluorescent moiety by a chemical agent; photobleaching the fluorescent moiety; permanently and irreversibly extinguishing the fluorescent moiety; or quenching the fluorescent moiety.

In certain embodiments, the primary detectable probe does not comprise a detectable label. In certain embodiments, the primary detectable probe does not comprise a fluorescent moiety. In certain embodiments, the primary detectable probe binds to a detectably labeled probe. In certain embodiments, the primary detectable probe hybridizes to a detectably labeled probe comprising a fluorescent moiety.

In certain embodiments, one or more of the encoding probes comprise a circular or circularizable probe or probe set. In some instances, one or more of the encoding probes comprise a ligatable probe or probe set (e.g., ligated linear probe). In certain embodiments, the two ends of a circularizable probe or probe sets are ligated using DNA-templated ligation or RNA-templated ligation. In certain embodiments, the circularizable probe comprises one or more ribonucleotides, e.g., at a 3′ ligatable end. In certain embodiments, the circularizable probe is a padlock probe. In some embodiments, the circularizable probe is ligated (e.g., circularized) upon hybridization to the target nucleic acid, and the ligation is templated by the circularizable probe itself. For example, a circularizable probe may be a turtle probe, wherein a hairpin structure brings the two ends of the circularizable into close proximity on an internal ligation template, for example as described in Stougaard et al., (2007) BMC Biotechnol. 7, 69, the content of which is herein incorporated by reference in its entirety. In some embodiments, the one or more second encoding probes comprise one or more second hybridization regions complementary to one or more second target sequences in the biological sample. In certain embodiments, the second target sequence can be a continuous sequence or a split sequence comprising two or more subsequences in the same nucleotide molecule or in different variants of a nucleotide molecule (e.g. nucleotide molecules corresponding to different alleles of a gene). In certain embodiments, a second target sequence can be a continuous sequence in a genomic DNA, a cDNA, an mRNA, a viral RNA, or a non-coding RNA. In certain embodiments, the two or more subsequences are in multiple oligonucleotide probes that hybridize to a genomic DNA, a cDNA, an mRNA, a viral RNA, or a non-coding RNA.

In some embodiments, an encoding probe comprises a circular or circularizable probe. In some embodiments, an encoding probe comprising a circular or circularizable probe is provided as a single probe molecule. In some embodiments, an encoding probe comprising a circularizable probe is provided as two or more probe molecules, e.g. a circularizable probe set. In certain embodiments, a circularizable probe set comprises two or more probe molecules which may be connected (e.g., via enzymatic or chemical ligation) to form a single circular molecule probe. In certain embodiments, a circularizable probe set comprises two probe molecules each comprising a sequence that hybridizes to a subsequence of a target sequence, and the two probe molecules each further comprises a sequence that hybridizes to a subsequence of a splint. In some embodiments, the splint hybridizes to one or more barcode regions. In some embodiments, the splint hybridizes to a barcode region which is a split barcode region. A circularizable probe or probe set can be connected (e.g., via enzymatic or chemical ligation) using a target sequence as a template, with or without gap filling. Likewise, a circularizable probe or probe set can be connected (e.g., via enzymatic or chemical ligation) using a splint as a template, with or without gap filling. In some embodiments, a circularizable probe set comprises two or more probe molecules that are connected using a target sequence as template (e.g., to ligate probe sequences hybridized to a target sequence) and using one or more splints as template (e.g., to ligate barcode sequences in the probe molecules hybridized to the splint(s)). In some embodiments, the splint can serve as a primer for RCA using the circularized probe as template. In some embodiments, an RCA primer other than a splint can be used to prime the RCA. In some embodiments, the target nucleic acid can be used to prime the RCA.

In some embodiments, a circular or circularizable encoding probe is used to generate an RCA product in the biological sample. In certain embodiments, the RCA product can be detected by a secondary detectable probe. In certain embodiments, upon contacting the biological sample with a plurality of secondary detectable probes, the secondary detectable probes bind (e.g. hybridize) to a complementary sequence of one or more barcode regions. In certain embodiments, the secondary detectable probes are cleavable or degradable. In certain embodiments, the contacting of the biological sample by the secondary detectable probes and the detection of signal associated with the secondary detectable probe are performed sequentially, e.g., in sequential detectable probe hybridization cycles, wherein in each cycle, the RCA products in the biological sample are contacted with one or more secondary detectable probes which are detected.

In certain embodiments, the secondary detectable probe is not circularized upon hybridization to the RCA product. In certain embodiments, the secondary detectable probe is circularized upon hybridization to the RCA product. In certain embodiments, this circularized secondary detectable probe is not used as a template to generate an RCA product for detection. In certain embodiments, this circularized secondary detectable probe is used as a template to generate an RCA product for detection.

In certain embodiments, the secondary detectable probe is provided as a single probe molecule. In certain embodiments, the secondary detectable probe is provided as multiple probe molecules which may but do not need to be connected (e.g., via enzymatic or chemical ligation) to form a single probe molecule.

In certain embodiments, the secondary detectable probe comprises a detectable label. In certain embodiments, the secondary detectable probe comprises a fluorescent moiety. In certain embodiments, a signal associated with the fluorescent moiety can be extinguished by unhybridizing the secondary detectable probe from the RCA product; enzymatically cleaving the fluorescent moiety from the secondary detectable probe; masking, removing or modifying the fluorescent moiety; cleaving a linker linking the fluorescent moiety to the secondary detectable probe; chemically or photochemically modifying the fluorescent moiety; bleaching the fluorescent moiety by a chemical agent; photobleaching the fluorescent moiety; permanently and irreversibly extinguishing the fluorescent moiety; or quenching the fluorescent moiety.

In certain embodiments, the secondary detectable probe does not comprise a detectable label. In certain embodiments, the secondary detectable probe does not comprise a fluorescent moiety. In certain embodiments, the secondary detectable probe binds to a detectably labeled probe. In certain embodiments, the secondary detectable probe hybridizes to a detectably labeled probe comprising a fluorescent moiety.

In some embodiments, a detectable probe comprising a detectable label (e.g., a fluorescent moiety) can be pre-hybridized to an intermediate probe (e.g., an L-probe disclosed herein) prior to contacting a biological sample. In some aspects, pre-hybridization of detectable probes to intermediate probes can improve the efficiency of in vitro hybridization and removal of the detectable probes. In some aspects, pre-hybridization of detectable probes can reduce the concentration and/or amount of unbound detectable probes that are added to a biological sample, which can reduce background fluorescence caused by unspecific binding of detectable probes.

In some embodiments, the first encoding probe and the one or more second encoding probes can be contacted with the biological sample in any suitable order. For instance, the probes can be added separately (e.g., provided in a separate solution) to contact the biological sample, but the contact can be simultaneous and optionally followed by a step of removing probe molecules that do not hybridize to the first or second target sequences in the biological sample. In other examples, the probes can be pre-combined (e.g., pre-mixed in the same solution) and added together to the biological sample, optionally followed by a step of removing probe molecules that do not hybridize to the first or second target sequences in the biological sample. In still other examples, the biological sample contacts the first encoding probe, optionally followed by a step of removing probe molecules that do not hybridize to the first target sequence in the biological sample, and then the biological sample contacts the one or more second encoding probes, optionally followed by a step of removing probe molecules that do not hybridize to a second target sequence in the biological sample. In yet other examples, the biological sample contacts the one or more second encoding probes, optionally followed by a step of removing probe molecules that do not hybridize to the second target sequence in the biological sample, and then the biological sample contacts the first encoding probe, optionally followed by a step of removing probe molecules that do not hybridize to the first target sequence in the biological sample.

In some aspects, the methods disclosed herein comprise a plurality of encoding probes that hybridize to the target nucleic acid. In some aspects, the methods disclosed herein comprise at least about 3, at least about 5, at least about 10, at least about 15, at least about 20, at least about 25, at least about 30, or at least about 35 encoding probes that hybridize to the target nucleic acid, where each probe comprises a hybridization region that hybridizes to a target sequence of the target nucleic acid.

In some embodiments, a nucleic acid probe disclosed herein may comprise 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the nucleic acid probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of probes (e.g., the encoding probes) is less than the number of distinct targets (e.g., target sequences) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes.

In some embodiments, a method disclosed herein comprises multiplexed analysis of a biological sample comprising consecutive cycles of detectable probe hybridization, fluorescence imaging, and signal removal. Exemplary detectable reactive reagents and methods are described in U.S. Pat. No. 6,828,109, US 2019/0376956, US 2019/0376956 A1, US 2022/0026433 A1, US 2022/0128565 A1, and US 2021/0222234 A1, all of which are incorporated herein by reference in their entireties.

In some embodiments, a method disclosed herein comprises nucleic acid sequencing, including in situ detection (e.g., in situ sequencing and/or sequential detectable probe hybridization) in a sample, which typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ detection are described, for example, in Mitra et al., (2003) Anal. Biochem. 320, 55-65, and Lee et al., (2014) Science, 343(6177), 1360-1363, each of which are herein incorporated by reference in their entireties. In addition, examples of methods and systems for performing in situ detection are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequence detection comprise, but are not limited to, STARmap (described for example in Wang et al., (2018) Science, 361(6499) 5691, the content of which is herein incorporated by reference in its entirety), MERFISH (described for example in Moffitt, (2016) Methods in Enzymology, 572, 1-49, the content of which is herein incorporated by reference in its entirety), hybridization-based in situ sequencing (HybISS) (described for example in Gyllborg et al., Nucleic Acids Res (2020) 48(19):e112, the content of which is herein incorporated by reference in its entirety), and FISSEQ (described for example in US 2019/0032121, the content of which is herein incorporated by reference in its entirety).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequence analysis of the target and/or barcoded probes can be performed by sequential hybridization (e.g., sequencing by hybridization and/or sequential in situ fluorescence hybridization). Sequential fluorescence hybridization can involve sequential hybridization of detection probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and WO 2021/138676, all of which are incorporated herein by reference in their entireties.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization can be used for sequencing or sequence detection. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), both of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

(a) Hybridization and Ligation

In some embodiments, a hybridization product is formed comprising the pairing of substantially complementary or complementary nucleic acid sequences. In some embodiments, the hybridization product or complex is formed comprising at least two different molecules or three different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets (e.g., encoding probes or probe sets; linear probe sets; circular probes or circularizable probes or probe sets as described herein) can be hybridized to an endogenous analyte and/or a labeling agent (e.g., reporter oligonucleotide) and each probe may comprise one or more barcode sequences or regions.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, the ligase is a circligase.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead ligation occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g. padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, such as steps comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(b) Primer Extension and Amplification

In some embodiments, a product that is a primer extension product can be generated using the various probes and probe sets (e.g., circular probes or circularizable probes or probe sets) described herein.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. In some embodiments, a primer extension reaction is any method where two nucleic acid sequences become linked (e.g., hybridized) by an overlap of their respective terminal complementary nucleic acid sequences (for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, an amplification product of one or more polynucleotides, for instance, a circular probe or circularizable probe or probe set is generated. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof. In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C. In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, branched RCA, dendritic RCA, and combinations thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801, all of which are herein incorporated by reference in their entireties). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase. In some embodiments, rolling circle amplification products can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification. In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modifications and polymer matrixes that can be employed in accordance with the provided embodiments include those described in, for example, US 2016/0024555, US 2018/0251833, and US 2017/0219465, the entire contents of each of which are incorporated herein by reference. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures. The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress. In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

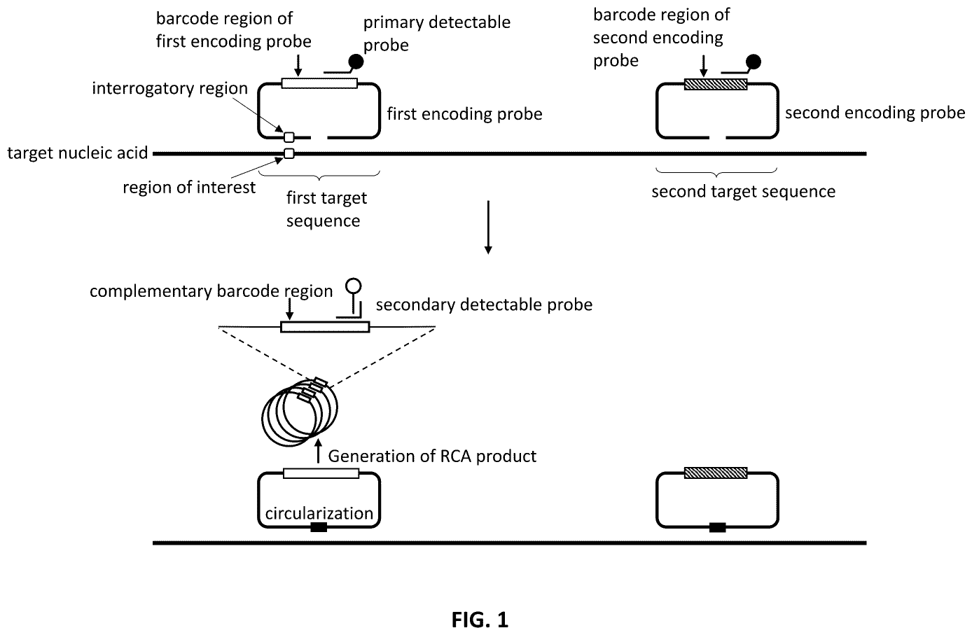

In some embodiments, a product herein includes a molecule or a complex generated in a series of reactions, e.g., hybridization, ligation, extension, replication, transcription/reverse transcription, and/or amplification (e.g., rolling circle amplification), in any suitable combination, generated using the probes described herein. For example, a product comprising a target sequence for a probe disclosed herein (e.g., a target nucleic acid as shown in FIG. 1) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., encoding probe barcode regions as shown in FIGS. 1 and 2B). In other examples, a product comprising a target sequence for a probe disclosed herein (e.g., the RCA product of an encoding probe, as shown in FIG. 1) may be an RCA product of a circularizable probe or probe set which can be hybridized by a detectable probe. In other examples, a product comprising a target sequence for a probe disclosed herein (e.g., a secondary detectable probe as shown in FIG. 1) may a probe hybridizing to an RCP. The probe may comprise an overhang that does not hybridize to the RCP but hybridizes to another probe.

FIG. 1 shows a schematic illustrating an exemplary configuration of a first encoding probe and a second encoding probe hybridized to a first and second target sequence, respectively, of a target nucleic acid. The first encoding probe comprises an interrogatory region for interrogating a region of interest in the first target sequence. Each of the encoding probes comprises a barcode region comprising one or more barcode sequences corresponding to the target nucleic acid and/or a sequence thereof (e.g., the first or second target sequence). The barcode region in the first encoding probe may comprise a barcode sequence corresponding to a sequence in the region of interest in the first target sequence. The barcode region may comprise barcode sequences, e.g., one or more hybridization barcode sequences (e.g. for detection by FISH, such as single molecule FISH (smFISH)), that can be detected in sequential hybridization cycles to decode the target nucleic acid or sequence thereof, and/or one or more amplifiable barcode sequences (corresponding to the first target sequence or a portion thereof and/or the target nucleic acid or a portion thereof) or further amplifiable barcode sequences (corresponding to the region of interest) that can be detected using in situ detection (e.g., in situ sequencing and/or sequential detectable probe hybridization, e.g., in situ sequencing by ligation, sequencing by synthesis, or sequencing by hybridization). In the same encoding probe, the hybridization barcode sequence(s) and the amplifiable barcode sequence(s) can be distinct sequences or identical in sequence (e.g., in one or multiple identical copies of the same barcode sequence). Further, a hybridization barcode sequence and an amplifiable barcode sequence in the same encoding probe can physically overlap or be separated by a bond or intervening nucleotide residue(s). The barcode regions of the first and second encoding probes can comprise one or more common barcode sequences. Alternatively, the barcode regions of the first and second encoding probes may not comprise any common barcode sequence.

In any of the embodiments herein, multiple copies of a hybridization barcode sequence can be present in one or more of the encoding probes targeting the same target nucleic acid. In any of the embodiments herein, an amplifiable barcode sequence can be an amplified barcode sequence present in multiple copies for detection, e.g., in an RCA product as shown in FIG. 2A or in any of the branched structures as shown in FIG. 2B.

The encoding probes can be hybridized with primary detectable probes for generating a signal associated with the target nucleic acid (e.g., an smFISH signal). The first encoding probe can be circularizable (e.g., as shown in FIG. 1), and the second encoding probe can be circularizable (e.g., as shown in FIG. 1) or circular (not shown). The interrogatory region can be on one or both arms (e.g., regions that hybridize to the target nucleic acid) of the first encoding probe or a flap region connected to one arm. The interrogatory region can be (but does not need to be) at the 3′ end and/or the 5′ end of the first encoding probe. In some examples, the encoding probes are circularized (e.g., ligated using the target sequences as template) and the first encoding probe is used as template for a rolling circle amplification (RCA) reaction to generate an RCA product. The complement of the barcode region in the RCA product of the first encoding probe can be hybridized by a secondary detectable probe to generate a signal associated with the target nucleic acid or a sequence thereof (e.g., the region of interest). In some examples, an RCA product of the circular or circularized second encoding probe can be generated using the same RCA primer sequence or different RCA primer sequences. For instance, the first and second encoding probes can comprise a common amplifiable barcode sequence but different backbone sequences that allow different RCA primers to hybridize. In a particular example, signals associated with the common amplifiable barcode sequence are detected in the RCA product of the circularized first encoding probe generated using a first RCA primer (which does not amplify the circular or circularized second encoding probe), and if the signals are low in amplitude, the RCA product of the circular or circularized second encoding probe can be generated using a second RCA primer. The RCA products of the first and second encoding probes both carry multiple copies of the complement of the common amplifiable barcode sequence, and the RCA products can be detected using the same secondary detectable probes to increase the signal amplitude. As such, the signal amplitude is tunable.

FIG. 2A shows schematics illustrating sequential detection of smFISH and RCA signals from a target nucleic acid using a first encoding probe and a plurality of second encoding probes. The first encoding probe and second encoding probes are hybridized to the target nucleic acid at a first target sequence and a plurality of second target sequences, respectively. Each encoding probe comprises a barcode region. In some examples, the first encoding probe comprises a barcode region that comprises one or more different barcode sequences from the barcode sequence(s) in the barcode regions of the second encoding probes. Likewise, the barcode regions of the plurality of second encoding probes may comprise different barcode sequences or one or more common barcode sequences. As shown in panel (i), the encoding probes can be hybridized with primary detectable probes for generating signals associated with the target nucleic acid (e.g., smFISH signals), for instance, in sequential hybridization cycles using various primary detectable probes or combinations thereof. As shown in panel (ii), the encoding probes can be circularized (e.g., ligated using the target sequences as template) and the first encoding probe is used as template for a rolling circle amplification (RCA) reaction to generate an RCA product, e.g., using an RCA primer that hybridizes to the first encoding probe. If an RCA primer is used, the RCA primer may hybridize to any suitable region of the first encoding probe, for example a barcode region of the first encoding probe, or a region of the encoding probe that is not in the barcode region. The RCA primer may be specific to the first encoding probe, or common to the first encoding probe and one or more second encoding probes. The RCA primer may be specific to one or more encoding probes targeting a first target nucleic acid, or common to one or more encoding probes targeting a first target nucleic acid and one or more other encoding probes targeting a second target nucleic acid. In some embodiments, the target nucleic acid or a portion thereof can be used to prime the RCA reaction (e.g., using target-primed RCA). The complement of the barcode region in the RCA product of the first probe is hybridized by a secondary detectable probe to generate a signal associated with the target nucleic acid or a sequence thereof (e.g., the first target sequence which may comprise a region of interest). As shown in panel (iii), RCA products can be generated and detected from the second target sequences, thereby generating a second signal associated with the target nucleic acid or a sequence thereof (e.g., the second target sequence which may comprise one or more additional regions of interest).

FIG. 2B shows schematics illustrating various encoding probe or encoding probe set designs for signal amplification and detection. Each encoding probe or probe set hybridizes to a target sequence in a target nucleic acid. Hybridized encoding probes or probe sets can be ligated at one or more ends to form a ligated probe, which may be linear or circular. Encoding probes may comprise a region (e.g., an overhang or barcode region), for hybridizing and/or binding directly or indirectly to one or more detectable probes. Ligated encoding probes can form an amplification complex (e.g., a branched complex, for example as described in Section V) with detectable probes. Detectable probes may be directly labeled, or hybridized to probes comprising detectable labels (e.g. fluorophores). Detectable probes can comprise a nucleic acid probe that hybridizes to an encoding probe (e.g., at a barcode sequence), and/or a nucleic acid probe that hybridizes to a detectable probe that in turn hybridizes to an encoding probe (e.g., at a barcode sequence). A detectable probe can comprise one or more barcode sequences in a region that does not hybridize to the encoding probe. Likewise, a detectable probe can comprise one or more barcode sequences in a region that does not hybridize to the detectable probe that is hybridized to the encoding probe. Multiple copies of the same barcode sequence can be present in the branched structure comprising multiple detectable probes hybridized to the same detectable probe which is hybridized to the encoding probe, e.g., as shown in FIG. 2B, and the barcode sequence can be viewed as an amplified barcode sequence for detection (e.g., by binding to a fluorescently labeled probe). Encoding probe sets may be provided as a first and second linear probe, and each linear probe can comprise a region for hybridizing one or more secondary or higher order nucleic acid probes, which may be detectable probes. Any of the target nucleic acids shown in FIG. 2B can be an RNA and any one or more of the encoding probes can comprise one or more ribonucleotides, e.g., at a 3′ ligatable end prior to the ligation.

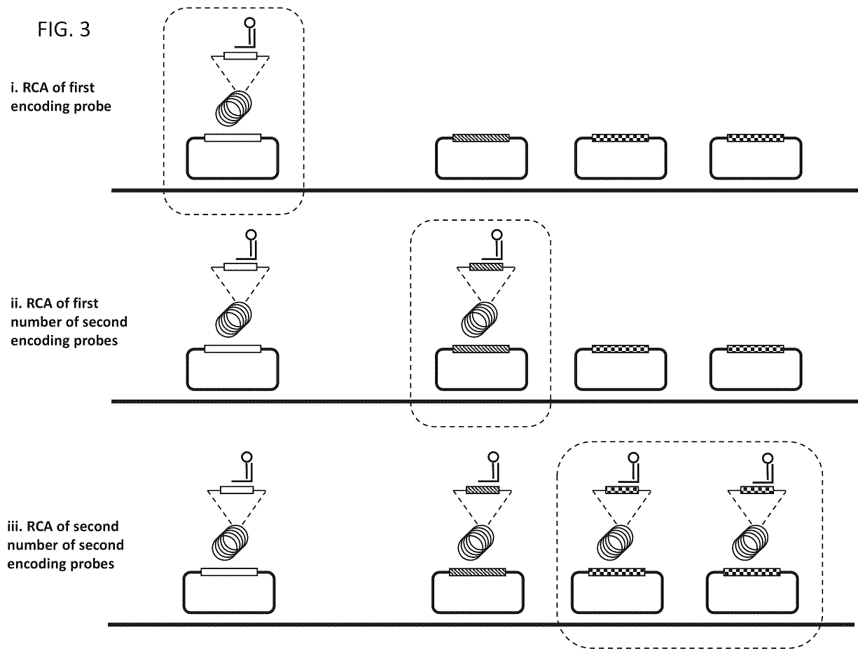

FIG. 3 shows schematics illustrating exemplary sequential detection of smFISH and RCA signals from a target nucleic acid using a first encoding probe, a first number of second encoding probes, and a second number of second encoding probes. Prior to step (i), the encoding probes are hybridized by primary detectable probes to generate signals (e.g., smFISH signals) that can be detected and decoded to detect the target nucleic acid. The smFISH signals may but do not need to provide sufficient information to detect and/or decode the target nucleic acid. However, the amplitude of the smFISH signals may be used to guide the selection of how many and/or which of the second encoding probes to amplify by RCA. In step (i), an RCA product of the first encoding probe is generated and a first signal is detected. The first signal may be the signal in one hybridization cycle or may comprise a series of signals in sequential hybridization cycles. The first signal may but does not need to provide sufficient information to detect and/or decode the target nucleic acid or a sequence thereof. However, the amplitude of the first signal may be used to guide the selection of the first number and/or the second number and/or which of the second encoding probes to amplify, if step (ii) and/or step (iii) are needed. In step (ii), RCA product(s) of the first number of second encoding probes is/are generated and detected. In step (iii), RCA product(s) of a second number of second encoding probes is/are generated and detected. The RCA products in steps (i)-(iii) may comprise complements of the same barcode sequences or different barcode sequences (e.g., as shown in FIG. 3). Steps (i)-(iii) may be performed in any suitable order, and signal amplitude from one step may be used to determine whether a subsequent step is necessary, and if so, how many and/or which encoding probes may be amplified by RCA and detected. Information from the smFISH signals and the RCA signals in any one or more of steps (i)-(iii) can be correlated and/or compared to analyze the target nucleic acid or one or more sequences thereof.

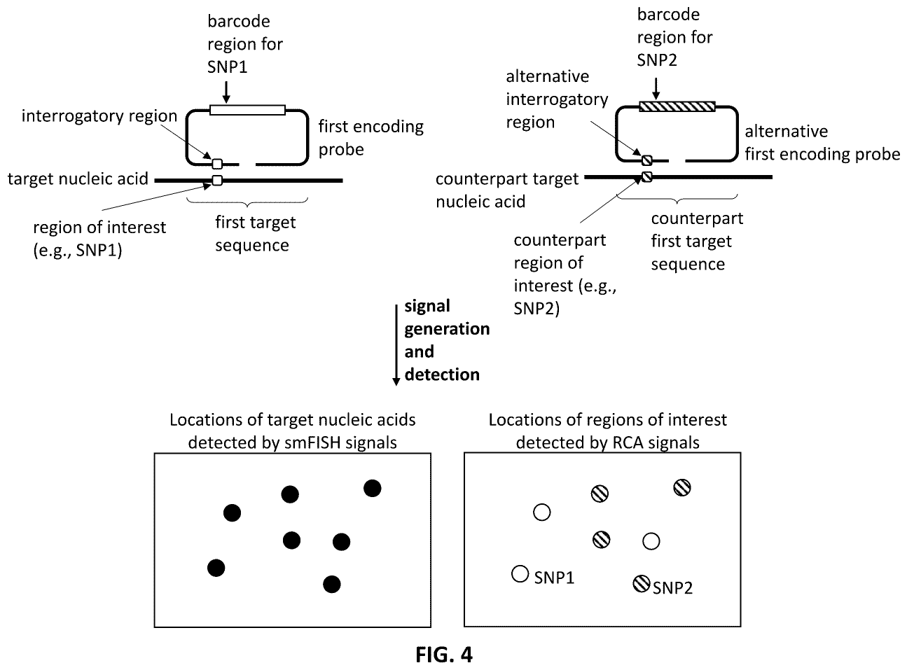

FIG. 4 shows a schematic illustrating an exemplary configuration of a first encoding probe hybridized to a first target sequence comprising a region of interest (e.g., SNP1) in a target nucleic acid, and an alternative first encoding probe hybridized to a counterpart first target sequence comprising a counterpart first region of interest (e.g., SNP2) in a counterpart target nucleic acid. The region of interest and counterpart region of interest may be different alleles of a single nucleotide polymorphism (SNP) or alleles of different SNPs. Each encoding probe comprises a barcode region which comprises a barcode sequence corresponding to the region of interest to which it hybridizes. In cases where the regions of interest are alleles of the same SNP, each target nucleic acid may comprise the same second target sequences (not shown), which are hybridized by a common set of second encoding probes. The first, alternative first, and second encoding probes can be hybridized by primary detectable probes (e.g., smFISH probes), which are detected to generate signals (e.g., smFISH signals) that can be used to decode and detect the target nucleic acid and counterpart target nucleic acid (e.g., including molecules of the target nucleic acid comprising different alleles of the same SNP) at locations in the sample. RCA can be performed for the first encoding probe and alternative first encoding probe, and different secondary detectable probes are hybridized to the complements of the barcode regions in the RCA products. The RCA products are detected to identify the region of interest and counterpart region of interest (e.g., SNP1 and SNP2) at locations in the sample. The smFISH readout and the RCA-based readout can be correlated and/or compared. In some instances, the encoding probe comprises one or more ribonucleotides in the interrogatory region for interrogating a region of interest in the target sequence.

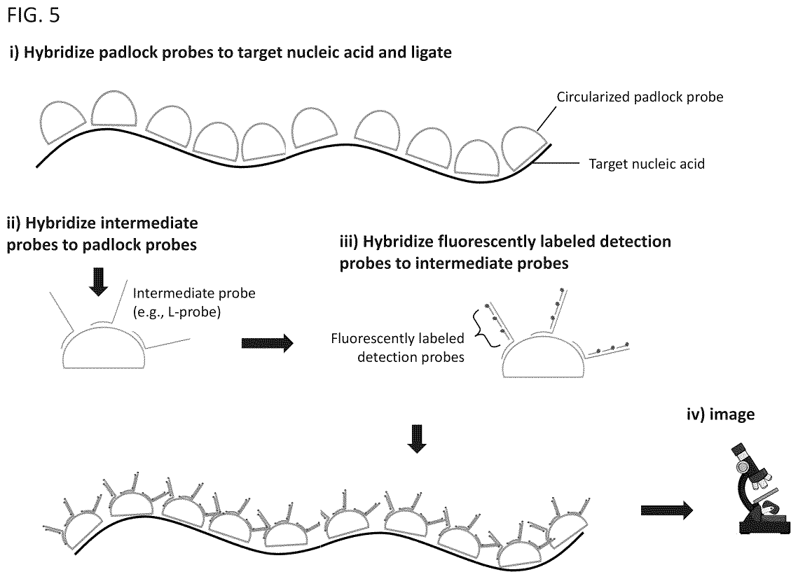

FIG. 5 shows an exemplary workflow for detecting a signal associated with a target nucleic acid in a biological sample using a plurality of circularizable probes (e.g., padlock probes, serving as encoding probes as described herein). Padlock probes can be hybridized to and circularized on a target nucleic acid in step i) and each padlock probe can be hybridized with one or more secondary probes (e.g., L-probes, serving as primary detectable probes as described herein) in step ii). L-probes (which may optionally be fluorescently labeled) can be hybridized with fluorescently labeled detection probes in step iii), and the sample can be imaged to detect smFISH signals associated with the L-probes and the fluorescently labeled detection probes hybridized thereto. After signal detection, the L-probes and the fluorescently labeled detection probes can be removed, and another set of L-probes and fluorescently labeled detection probes can be hybridized to the padlock probes. The L-probe/fluorescently labeled detection probe hybridization can be performed in sequential cycles to generate a signal code sequence comprising signals from the sequential cycles for decoding the target nucleic acid. In some aspects, the exemplary workflow shown in FIG. 5 may be performed with any suitable encoding probes or probe sets, e.g., as depicted in FIG. 1, FIGS. 2A-2B, FIG. 3, or FIG. 4.

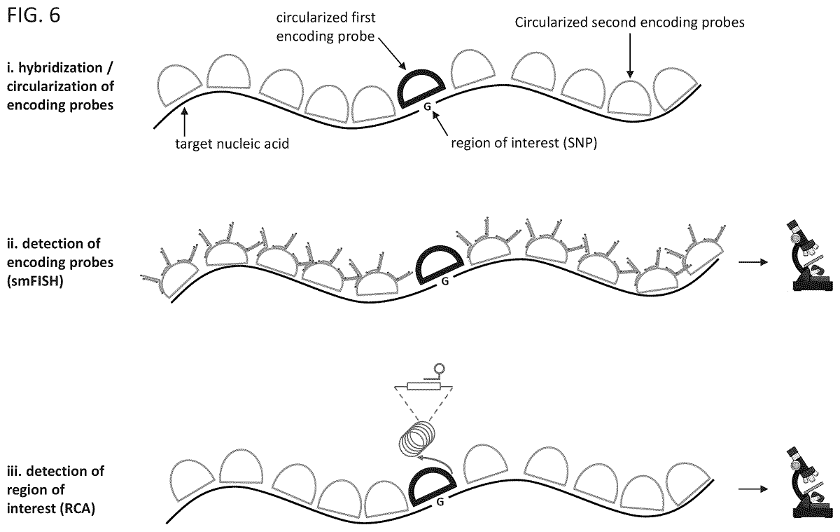

FIG. 6 shows an exemplary workflow for detecting a signal associated with a target nucleic acid, and a signal associated with a region of interest in the target nucleic acid, in a biological sample. As shown in panel (i), a plurality of circularizable probes (e.g., padlock probes as encoding probes described herein) can be hybridized to a target nucleic acid and circularized. A first encoding probe hybridizes to a first target sequence comprising a region of interest (e.g., “G” at an SNP), and a plurality of second encoding probes hybridize to a plurality of second target sites. As shown in panel (ii), the second encoding probes (and optionally the first encoding probe) can be labeled with intermediate probes (e.g., L-probes serving as primary detectable probes as described herein) and fluorescently labeled detection probes that hybridize the intermediate probes, and the sample is imaged to detect signals (e.g., smFISH signals) associated with the target nucleic acid. In this example, the second encoding probes are not enzymatically amplified before detection. As shown in panel (iii), the intermediate probes (e.g., L-probes comprising an overhang that is for binding to the detectable probes) and fluorescently labeled detection probes can be stripped from the encoding probes (which remain hybridized to the target nucleic acid) and/or the signals associated with the intermediate probes and fluorescently labeled detection probes can be extinguished. An RCA primer can be hybridized to the first encoding probe to perform an RCA reaction using the first encoding probe as template to generate an RCA product. The RCA product can be hybridized by detectable probes (e.g., fluorescently labeled probes and/or probes (e.g., L-probes) that can hybridize to fluorescently labeled probes) and the sample is imaged to detect signals associated with the region of interest.

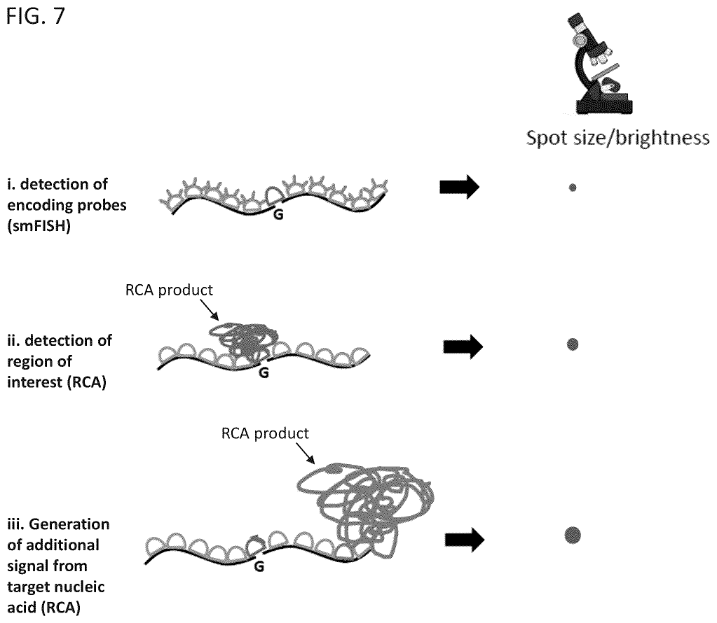

FIG. 7 shows an exemplary workflow for generating and detecting tunable signals associated with a target nucleic acid and a region of interest in the target nucleic acid in a biological sample. In steps (i) and (ii) the target nucleic acid and region of interest are detected as in FIG. 6. In step (iii), an RCA product is generated using a first number of the second encoding probes to generate a larger and/or higher intensity signal than the signals generated in step (i) and/or (ii). A larger intensity and/or higher intensity signal may be generated, for example, by generating a greater number of RCA products by amplifying more circular or circularized probes in step (ii) and/or by performing an RCA reaction for a longer duration, thereby generating longer RCA product(s). In both cases, more copies of a barcode region or complement thereof can be created for detection by detectable probes (e.g. the secondary detectable probes).

### V. Detection and Analysis

In some aspects, after formation of a hybridization complex comprising nucleic acid probes and/or probe sets described in Section III and further processing (e.g., ligation, extension, amplification, or any combination thereof), for example as described in Section II and Section IV, the method further includes detection of one or more of the encoding probes hybridized to the target nucleic acid or any products generated therefrom or a derivative thereof. In any of the embodiments herein, the method can further comprise imaging the biological sample to detect a ligation product or a circularized encoding probe or product thereof. In any of the embodiments herein, a sequence of the ligation product, rolling circle amplification product, or other generated product can be analyzed in situ in the biological sample. In any of the embodiments herein, the imaging can comprise detecting a signal associated with a fluorescently labeled probe that directly or indirectly binds to a rolling circle amplification product of one or more circularized encoding probe. In any of the embodiments herein, the sequence of the ligation product, rolling circle amplification product, or other generated product can be analyzed by sequential hybridization, sequencing by hybridization, sequencing by ligation, sequencing by synthesis, sequencing by binding, or a combination thereof.

In any of the embodiments herein, a sequence associated with the target nucleic acid or the encoding probe(s) can comprise one or more barcode sequences or complements thereof. In any of the embodiments herein, the sequence of the rolling circle amplification product can comprise one or more barcode sequences or complements thereof. In any of the embodiments herein, a ligated probe can comprise one or more barcode sequences or complements thereof. In any of the embodiments herein, the one or more barcode sequences can comprise a barcode sequence corresponding to the target nucleic acid. In any of the embodiments herein, the one or more barcode sequences can comprise a barcode sequence corresponding to the region of interest, such as variant(s) of a single nucleotide of interest or a single nucleotide polymorphism (SNP).

In any of the embodiments herein, a detecting step can comprise contacting the biological sample with one or more detectably-labeled probes that hybridize to the rolling circle amplification product, and dehybridizing the one or more detectably-labeled probes from the rolling circle amplification product. In any of the embodiments herein, the contacting and dehybridizing steps can be repeated with the one or more detectably-labeled probes and/or one or more other detectably-labeled probes that hybridize to the rolling circle amplification product.

In any of the embodiments herein, the detecting step can comprise contacting the biological sample with one or more primary detectable probes that directly hybridize to one or more of the plurality of encoding probes. In some instances, the detecting step can comprise contacting the biological sample with one or more primary detectable probes that indirectly bind (e.g. hybridize) to one or more of the plurality of encoding probes. In some instances, the detecting step can comprise contacting the biological sample with one or more primary detectable probes that directly bind (e.g. hybridize) to one or more of the plurality of encoding probes. In any of the embodiments herein, the detecting step can comprise contacting the biological sample with one or more primary detectable probes that directly or indirectly bind (e.g. hybridize) to one or more of the encoding probes. In any of the embodiments herein, the detecting step can comprise contacting the biological sample with one or more primary detectable probes that hybridize to one or more encoding probes (prior to performing an amplification reaction). In any of the embodiments herein, the detecting step can comprise a secondary detectable probe that directly or indirectly binds (e.g. hybridizes) to the rolling circle amplification product generated using one or more encoding probe.

In any of the embodiments herein, the detecting step can comprise contacting the biological sample with one or more secondary probes (e.g., secondary detectable probes) that directly or indirectly binds (e.g. hybridizes) to the rolling circle amplification product, wherein the one or more secondary probes are detectable using one or more detectably-labeled probes. In any of the embodiments herein, the detecting step can further comprise dehybridizing the one or more secondary probes and/or the one or more detectably-labeled probes from the rolling circle amplification product. In any of the embodiments herein, the contacting and dehybridizing steps can be repeated with the one or more secondary probes, the one or more detectably-labeled probes, one or more other secondary probes, and/or one or more other detectably-labeled probes.

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc., depending on the application.

In some embodiments, a method disclosed herein may also comprise one or more signal amplification components. In some embodiments, the present disclosure relates to the detection of nucleic acids sequences in situ using probe hybridization and generation of amplified signals associated with the encoding probes (e.g., described in Section III), wherein background signal is reduced and sensitivity is increased. In some embodiments, the RCA product generated using a method disclosed herein can be detected with a method that comprises signal amplification. In some embodiments, signal amplification may comprise use of one or more of the plurality of encoding probes.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is herein incorporated by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is herein incorporated by reference in its entirety), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956 A1, US 2022/0026433 A1, US 2022/0128565 A1, and US 2021/0222234 A1, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721, all of which are herein incorporated by reference in their entireties. See also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401, all of which are herein incorporated by reference in their entireties. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., US 2022/0064697 A1, the content of which is herein incorporated by reference in its entirety), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and directly or indirectly binds (e.g. hybridizes) to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes an assembly for branched signal amplification. In some embodiments, the assembly complex comprises an amplifier directly or indirectly bound, e.g. hybridized, (via one or more oligonucleotides) to a sequence of the probes (e.g., any described in Section III). In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the probes (e.g., any described in Section III) can be detected with a method that comprises signal amplification by performing a primer exchange reaction (PER). In various embodiments, a primer with a domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a plurality of concatemer primers and a plurality of labeled probes. see e.g., U.S. Pat. Pub. No. US20190106733, which is incorporated herein by reference in its entirety, for exemplary molecules and PER reaction components.

In some embodiments, the methods comprise determining the sequence of all or a portion of the amplification product, such as one or more barcode sequences present in the amplification product, e.g., by sequencing or by sequential detectable probe hybridization.

In some embodiments, the product or derivative of a first and second probe ligated together after hybridizing to the target nucleic acid can be analyzed by sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference in its entirety, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise detecting the amplification product (e.g., amplicon) and/or encoding probes hybridized to the target nucleic acid, for example, via binding of a detectable probe and detecting a detectable label comprised by the detectable probe. In some embodiments, the detectable probe comprises a detectable label that can be measured and quantitated. In some embodiments, a label or detectable label is a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, a fluorophore is a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. In some embodiments, “autofluorescence” is a general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s), probes, and/or amplification products (e.g., amplicon or RCA products) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Nucleotides having other fluorophores can be custom synthesized (See, e.g., Henegariu et al. (2000) Nature Biotechnol. 18:345, the content of which is herein incorporated by reference in its entirety).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (See, e.g., Lakowicz et al. (2003) Bio Techniques 34:62, the content of which is herein incorporated by reference in its entirety).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, an antibody is an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537, 4,849,336, and 5,073,562, the entire contents of each of which are incorporated herein by reference. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectable probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detectable probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, all of which are herein incorporated by reference in their entireties. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing comprise, but are not limited to, STARmap (described for example in Wang et al., (2018) Science, 361(6499) 5691, the content of which is herein incorporated by reference in its entirety), MERFISH (described for example in Moffitt, (2016) Methods in Enzymology, 572, 1-49, the content of which is herein incorporated by reference in its entirety), hybridization-based in situ sequencing (HybISS) (described for example in Gyllborg et al., Nucleic Acids Res (2020) 48(19):el 12, the content of which is herein incorporated by reference in its entirety), and FISSEQ (described for example in US 2019/0032121, the content of which is herein incorporated by reference in its entirety). In some cases, sequencing can be performed after the analytes are released from the biological sample.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, the barcodes of the probes (e.g., encoding probes) or complements or products thereof are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., detection oligonucleotides or detectable probes). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):el 12; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VI. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have any suitable alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Any suitable useful non-native bases that can be included in a nucleic acid or nucleotide can be used. See, for example, Ochoa and Milam, Molecules, 25(20):4659 (2020); and McKenzie et al., Chem Soc Rev., 50(8):5126-5164 (2021), the entire contents of each of which are incorporated herein by reference.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

Two nucleic acid sequences can become linked (e.g., hybridized) by an overlap of their respective complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

### (ix) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, MA), and Ampligase™ (available from Epicentre Biotechnologies, Madison, WI). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, a probe, or composition to be detected. For example, detectable labels can be incorporated during nucleic acid polymerization or amplification (e.g., Cy5®-labeled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™ Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families can provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLES

The following examples are included for illustrative purposes only and are not intended to limit the scope of the present disclosure.

### Example 1: Detection of Nucleic Acids In Situ Using Padlock Probes without Performing Rolling Circle Amplification (RCA)

This Example discloses exemplary methods for in situ detection of target nucleic acids using circularizable probes. In some aspects, single molecule fluorescent in situ hybridization (smFISH) assays are highly sensitive and are suited for the analysis of low-level analytes, such as transcripts of low expressed genes in a sample. However, smFISH signals may have intensities below an optimum range for detection. Furthermore, smFISH assays generally do not facilitate detection of variations in target nucleic acids at the level of individual nucleotides, such as single-nucleotide polymorphisms (SNPs).

This Example discloses exemplary methods for performing smFISH using circularizable probes (e.g., padlock probes), to detect a target nucleic acid. The method is compatible with further performing rolling circle amplification (RCA) of the circularizable probes (after being circularized) to generate a higher intensity signal for the same target nucleic acid, or to generate a detectable signal associated with a region of interest in the target nucleic acid, such as a SNP.

Mouse embryonic fibroblast 3T3 cells were fixed and prepared on slides for in situ hybridization. 30 different padlock probes targeting different sequences within a Polr2a transcript were added to the sample and allowed to hybridize at 37° C. (e.g. as illustrated in the schematic of FIG. 5). The sample was washed with formamide and SSC to remove unbound probes. Padlock probes were ligated (i.e. circularized) using SplintR Ligase. Intermediate probes (e.g., L-probes) serving as primary detectable probes as described herein, were hybridized to binding sites on the ligated padlock probes, and each padlock probe contained three intermediate probe binding sites. Each intermediate probe comprised a region for hybridizing to a padlock probe, and an overhang for binding fluorescently labeled detection probes. Fluorescently labeled detection probes comprising one or two fluorescent labels (e.g. single- and double-labeled detection probes) were hybridized to the intermediate probes. Samples were imaged by fluorescence microscopy to detect signals associated with the presence of Polr2a transcripts.

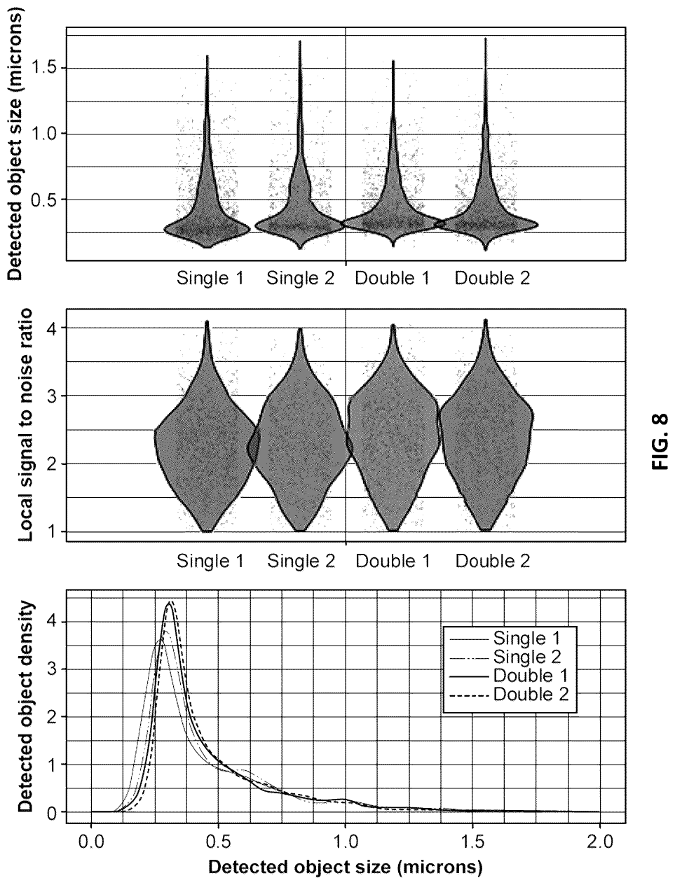

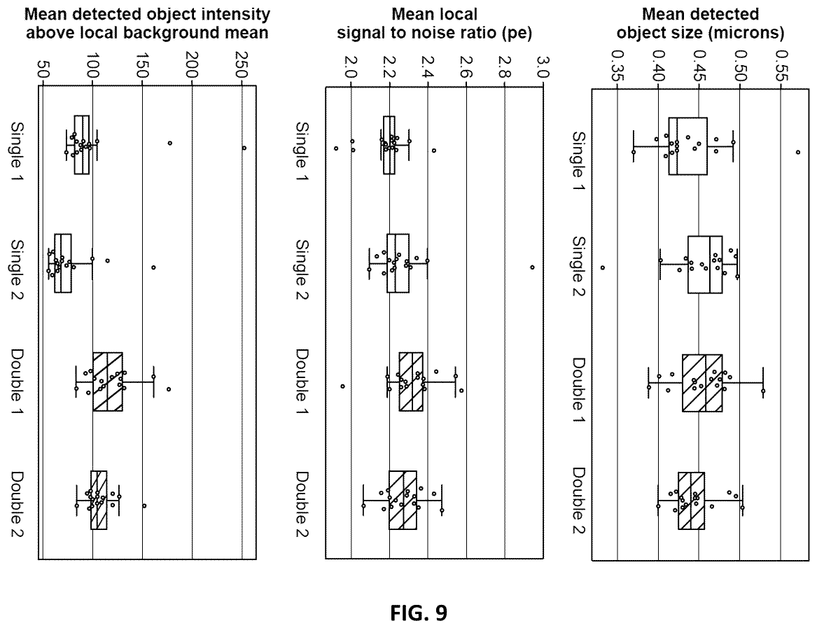

As shown in FIG. 8 and FIG. 9, signals corresponding to Polr2a transcripts were detected using both single-labeled and double-labeled detection probes and use of double-labeled detection probes facilitated generation of signals with higher intensity. In FIG. 8, detected object size, local signal to noise ratio, and detected object density are shown and results include two experimental replicates each for conditions with single-labeled (Single 1 and Single 2) and double-labeled (Double 1 and Double 2) detection probes. In FIG. 9, the results include two experimental replicates each for conditions with single-labeled (Single 1 and Single 2) and double-labeled (Double 1 and Double 2) detection probes, and the mean detected object size, mean local signal to noise ratio, and mean detected object intensity above local background are shown.

### Example 2: In Situ Detection of a First Signal and a Second Amplified Signal Corresponding to a Single Target Nucleic Acid Molecule

This Example discloses exemplary methods for performing smFISH using circularizable encoding probes to detect a target nucleic acid, and further performing rolling circle amplification (RCA) of the circularizable encoding probes (after being circularized) to generate a higher intensity signal for the same target nucleic acid.

A biological sample (e.g., fixed cells, a processed or cleared biological sample, a tissue sample, a sample embedded in a hydrogel, etc.) is contacted with a plurality of circular encoding probes or circularizable encoding probes or probe sets (e.g., a padlock probe, SNAIL probe, etc.). Each circularizable probe targets a different sequence within the same target nucleic acid (e.g. as shown in FIG. 6). In some examples, two or more of the circularizable encoding probes comprise different barcode regions and/or different primer binding sequences for RCA (e.g. as shown in FIG. 2A and FIG. 3). The probes are hybridized to the target sequences within a target nucleic acid, and the sample is washed to remove unbound probes. The circularizable encoding probes or probe sets are ligated to generate circularized encoding probes, and fluorescently labeled (e.g. Cy5-labeled) probes are bound (e.g. hybridized) directly or indirectly to the circularized probes, (e.g. as in Example 1). The sample is imaged by fluorescence microscopy to detect signals associated with the presence of the target nucleic acid. In some cases, the probes are ligated (e.g., circularized to form a circular probe or ligated to form a ligated probe that is not circular) using the target sequences as template. In some cases, the ligation can be performed before the sample is contacted with labeled probes (e.g., primary detectable probes). In other designs, the ligations may comprise a ligation external to the target sequence in a target nucleic acid. For instance, the ligations can comprise ligation templated on a DNA splint (which may but does not need to hybridize to the target nucleic acid), and/or ligation templated on a sequence of the probe itself (e.g., as in turtle probes).

To obtain a brighter signal from the target nucleic acid, a first number of circularized probes are selected for RCA (e.g. first encoding probes as shown in FIG. 2A and FIG. 3). The first number of circularized probes may comprise one, a subset, or all of the circularized encoding probes. One or more RCA primers are added to the sample, and hybridized to the first number of circularized probes via one or more primer binding sequences for RCA. An RCA reaction mixture (containing Phi29 reaction buffer, dNTPs, and Phi29 polymerase) is added to the sample. The sample is incubated at an incubation temperature (e.g., 30° C. or 37° C.) for a defined period of time (e.g. 3 hours), allowing the one or more circularized probes to be amplified by the DNA polymerase to generate an RCA product. The RCA reaction is terminated. Fluorescently labeled secondary detectable probes are hybridized to complements of barcode regions and/or primer binding sequences present in the RCA products. The sample is imaged by fluorescence microscopy to detect signals associated with the presence of the target nucleic acid.

In some examples, one or more additional rounds of RCA are performed using different subsets of the circularized encoding probes to obtain signals of different intensities (e.g. as shown in FIG. 3). In some cases, a subsequent RCA round is performed by hybridizing a different RCA primer to a subset of circularized encoding probes via a different primer binding sequence than used for a previous RCA round. The number of circularized probes selected for RCA and/or the duration of the RCA reaction in the first and subsequent rounds of RCA may be determined based on a desired signal intensity and/or the results of a previous round of imaging.

### Example 3: In Situ Detection of Signals Corresponding to a Target Nucleic Acid Molecule and a Region of Interest Therein

This Example discloses exemplary methods for performing smFISH using circularizable encoding probes to detect a target nucleic acid, and further performing rolling circle amplification (RCA) of a circularizable encoding probe (after being circularized) to detect a region of interest in the target nucleic acid.

A biological sample (e.g., fixed cells, a processed or cleared biological sample, a tissue sample, a sample embedded in a hydrogel, etc.) is contacted with a plurality of circularizable encoding probes or probe sets (e.g., a padlock probe, SNAIL probe, etc.). Each circularizable encoding probe targets a different sequence within the same target nucleic acid. The plurality of circularizable encoding probes comprises a circularizable first encoding probe which hybridizes to a first target sequence and comprises an interrogatory region for interrogating a region of interest (e.g. a SNP) in the first target sequence, and a plurality of circularizable second encoding probes which hybridize to a plurality of second target sequences (e.g. as shown in FIG. 2 and FIG. 6). The circularizable first and second encoding probes comprise different barcode regions and/or different primer binding sequences for RCA.

The encoding probes are hybridized to the target sequences, and the sample is washed to remove unbound probes. Encoding probes are ligated, and fluorescently labeled (e.g. Cy5-labeled) probes are bound (e.g. hybridized) directly or indirectly to circularized encoding probes, (e.g. as in Example 1). The sample is imaged by fluorescence microscopy to detect signals associated with the presence of the target nucleic acid.

To detect the region of interest, RCA is performed for the circularized first encoding probe using an RCA primer that is hybridized to a primer binding sequence or barcode region of the circularized first encoding probe. Fluorescently labeled secondary detectable probes are hybridized to complements of a barcode region and/or primer binding sequence present in the RCA product of the circularized first encoding probe. The sample is imaged by fluorescence microscopy to detect signals associated with the presence of the region of interest (e.g. SNP) in the target nucleic acid.

In some examples, one or more additional rounds of RCA are performed using one or more of the circularized second encoding probes to generate an additional and/or brighter signal associated with the target nucleic acid, e.g. as shown in FIG. 3 and FIG. 7.

In some examples, the biological sample comprises a counterpart target nucleic acid that comprises a counterpart region of interest. For example, the target nucleic acid and counterpart target nucleic acid may comprise the same transcript with different alleles of a SNP. In this example, a circularizable alternative first encoding probe may be included, which is hybridized to and circularized using the counterpart target nucleic acid, (e.g. as shown in FIG. 4). The circularized first encoding probe and circularized alternative first encoding probe comprise different barcode regions. To detect the region of interest and counterpart region of interest in different nucleic acids, RCA is performed for the circularized first encoding probe and circularized alternative first encoding probe and different fluorescently labeled probes are hybridized to the complements of the corresponding barcode regions in the RCA products. The sample is imaged to detect the locations of the different SNPs in the sample (e.g. as shown in FIG. 4).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
a) contacting the biological sample with a plurality of encoding probes, wherein:
the plurality of encoding probes comprises a first encoding probe and one or more second encoding probes,
the first encoding probe and each second encoding probe are capable of hybridizing to a first target sequence and a second target sequence, respectively, in a target nucleic acid in the biological sample,
the first encoding probe is circularizable and comprises an interrogatory region for interrogating a region of interest in the first target sequence, and
each second encoding probe is circular or circularizable;
b) circularizing the first encoding probe to generate a circularized first encoding probe, and optionally circularizing the one or more second encoding probes;
c) contacting the biological sample with one or more primary detectable probes that hybridize to the first encoding probe and/or the second encoding probe;
d) detecting a signal associated with the one or more primary detectable probes;
e) generating a rolling circle amplification (RCA) product of the circularized first encoding probe; and
f) detecting a signal associated with the RCA product.

2. The method of claim 1, wherein the interrogatory region is complementary to the region of interest, and hybridization of the interrogatory region to the region of interest allows ligation to circularize the first encoding probe using the first target sequence as a template.

3-6. (canceled)

7. The method of claim 1, wherein the first encoding probe comprises one or more ribonucleotides and/or the one or more second encoding probes comprise one or more ribonucleotides.

8-11. (canceled)

12. The method of claim 1, wherein the target nucleic acid comprises RNA.

13. The method of claim 1, wherein the biological sample comprises a counterpart target nucleic acid comprising a counterpart first target sequence having the same sequence as the first target sequence except that the region of the counterpart first target sequence corresponding to the region of interest is not complementary to the interrogatory region in the first encoding probe, thereby not allowing ligation of the ends of the first encoding probe using the counterpart target nucleic acid as a template.

14. The method of claim 1, wherein the first encoding probe comprises a first hybridization region complementary to the first target sequence or a portion thereof and the first hybridization region is a split hybridization region comprising a 5′ hybridization region and a 3′ hybridization region.

15-20. (canceled)

21. The method of claim 1, wherein each second encoding probe comprises a second hybridization region complementary to the second target sequence or a portion thereof.

22-24. (canceled)

25. The method of claim 1, wherein the first encoding probe and/or the one or more second encoding probes each independently comprise one or more barcode regions.

26. The method of claim 1, wherein the first encoding probe comprises one or more barcode sequences that are not present in the one or more second encoding probes.

27. The method of claim 1, wherein the one or more second encoding probes and/or the first encoding probe collectively comprise one or more hybridization barcode sequences that correspond to the target nucleic acid.

28-29. (canceled)

30. The method of claim 1, wherein the first encoding probe comprises an amplifiable barcode sequence, and the RCA product of the circularized first encoding probe comprises multiple copies of the complement of the amplifiable barcode sequence.

31-38. (canceled)

39. The method of claim 1, wherein in step b), the first encoding probe is circularized and the one or more second encoding probes are not circularized.

40-42. (canceled)

43. The method of claim 1, further comprising removing the one or more primary detectable probes without removing the first encoding probe and/or the one or more second encoding probes from the target nucleic acid.

44-45. (canceled)

46. The method of claim 1, wherein the RCA product of the first encoding probe is a first RCA product, and the circular or circularized one or more second encoding probes are used as templates to generate one or more second RCA products.

47. The method of claim 46, further comprising detecting a signal associated with the one or more second RCA products.

48. The method of claim 47, wherein the signal associated with the first RCA product and the signal associated with the one or more second RCA products are detected sequentially in either order.

49. The method of claim 1, wherein the RCA product is contacted with one or more secondary detectable probes that directly or indirectly binds to the RCA product.

50-56. (canceled)

57. The method of claim 1, wherein two or more of the second encoding probes comprise different primer binding sequences for RCA.

58. The method of claim 1, comprising using signals associated with the one or more primary detectable probes to select a duration of RCA to generate the RCA product of the first encoding probe.

59-78. (canceled)

79. The method of claim 1, wherein one or more of the signals are detected in situ in the biological sample.

80-83. (canceled)
